(19) United States
(12) Patent Application Publication
(10) Pub. No.: US 2013/0267597 A1
(43) Pub. Date: Oct. 10, 2013

(54) POLYUNSATURATED FATTY ACID-CONTAINING SOLID FAT COMPOSITIONS AND USES AND PRODUCTION THEREOF

(71) Applicant: DSM IP Assets B.V., Heerlen (NE)

(72) Inventors: S.P. Janaka Namal Senanayake, Overland Park, KS (US); Naseer Ahmed, Atlanta, GA (US); Jaouad Fichtali, Clarksville, MD (US)

(21) Appl. No.: 13/774,605

(22) Filed: Feb. 22, 2013

Related U.S. Application Data
(63) Continuation of application No. 12/201,728, filed on Aug. 29, 2008, now abandoned.
(60) Provisional application No. 60/969,539, filed on Aug. 31, 2007.

(51) Int. Cl. A23D 9/007; A23L 1/29; A61K 47/44

(52) U.S. Cl. CPC A23D 9/007; A61K 47/44; A23L 1/293
USPC 514/560; 426/607

(57) ABSTRACT

The present invention provides a solid fat composition that includes an oil having saturated fat and an oil having at least one long chain polyunsaturated fatty acid. In particular, the solid fat composition can have high levels of long chain polyunsaturated fatty acid and low to no presence of emulsifiers. In preferred embodiments, the polyunsaturated oil is an unwinterized microbial oil. The invention also relates to methods for making such compositions and food, nutritional, and pharmaceutical products comprising said compositions.

### CROSS-REFERENCE TO RELATED APPLICATIONS

This application claims the benefit of priority under 35 U.S.C. §119 to U.S. provisional patent application Ser. No. 60/969,536, filed Aug. 31, 2007, the entire contents of which are incorporated herein by reference.

### FIELD OF THE INVENTION

The invention relates to polyunsaturated fatty acid-containing solid fat compositions and uses and production thereof. The solid fat compositions of the present invention can include a microbially-derived long chain polyunsaturated fatty acid. The invention also relates to methods for making such products and food, nutritional, and pharmaceutical products comprising said compositions.

### BACKGROUND OF THE INVENTION

Dietary lipids are essential nutrients required for an overall healthful lifestyle. Lipids provide the most concentrated source of energy of any foods. The caloric value of lipids (9 kcal/g) is twice as high as that of proteins and carbohydrates (4 kcal/g). Lipids not only contribute to flavor, color, odor and texture of foods, but also confer a feeling of satiety after eating. Lipids also act as carriers of fat-soluble vitamins and supply essential fatty acids. The essential fatty acids are polyunsaturated fatty acids (PUFAs) with two or more double bonds in their backbone structure. There are two groups of essential fatty acids, the omega-3 fatty acids and the omega-6 fatty acids. Omega-3 PUFAs are recognized as important dietary compounds for preventing arteriosclerosis and coronary heart disease, for alleviating inflammatory conditions and for retarding the growth of tumor cells. Omega-6 PUFAs serve not only as structural lipids in the human body, but also as precursors for a number of factors in inflammation such as prostaglandins, and leukotrienes. An important class of both the omega-3 and the omega-6 PUFAs is long chain omega-3 and omega-6 PUFAs.

Fatty acids are classified as saturated and unsaturated fatty acids, the latter being further subdivided into monounsaturated and polyunsaturated fatty acids. Saturated fatty acids contain only single carbon-carbon bonds in the aliphatic chain and all other available bonds are taken up by hydrogen atoms. Unsaturated fatty acids contain carbon-carbon double bonds in the aliphatic chain. When an unsaturated fatty acid contains one carbon-carbon double bond in the molecule, it is called monounsaturated. PUFAs contain two or more carbon-carbon double bonds. Short chain fatty acids are about 2 to about 7 carbon atoms in length and medium chain fatty acids are about 8 to about 19 carbons in length. On the other hand, long chain fatty acids have from 20 to 24 or more carbons. Long chain PUFAs (LC-PUFAs) having 20 or more carbons are of particular interest in the present invention.

LC-PUFAs can be divided into two main categories depending on the position of the first double bond in the fatty acid carbon chain and are known as n-3 (or omega-3) and n-6 (or omega-6) families. The omega-3 or n-3 notation means that the first double bond in this family of PUFAs is three carbons from the methyl end of the molecule. The same principle applies to the omega-6 or n-6 notation. Of the LC-PUFAs, linoleic, linolenic, arachidonic, eicosapentaenoic, and docosahexaenoic acids containing respectively two, three, four, five, and six double bonds are of interest. Docosahexaenoic acid (“DHA”) has a chain length of 22 carbons with 6 double bonds beginning with the third carbon from the methyl end and is designated “22:6n-3”. Other important omega-3 LC-PUFAs include eicosapentaenoic acid (“EPA”) which is designated “20:5n-3,” and omega-3 docosapentaenoic acid (“DPA n-3”) which is designated “22:5n-3.” Important omega-6 LC-PUFAs include arachidonic acid (“ARA”) which is designated “20:4n-6,” and omega-6 docosapentaenoic acid (“DPA n-6”) which is designated “22:5n-6.”

The parent compounds of the omega-3 and omega-6 groups of fatty acids are linoleic acid (LA) and α-linolenic acid (ALA). LA and ALA are considered to be essential fatty acids for human health because humans cannot synthesize them and must obtain them from the diet.

Within the body, these parent compounds are metabolized by a series of alternating desaturations (in which an extra double bond is inserted by removing two hydrogen atoms) and elongations (in which two carbon atoms are added). This requires a series of special enzymes called desaturases and elongases. It is believed that the enzymes metabolizing both omega-6 and omega-3 fatty acids are identical, resulting in competition between the two PUFA families for these enzymes. Chain elongation and desaturation occurs only at the carboxyl end of the fatty acid molecule. Thus, all metabolic conversions occur without altering the omega end of the molecule that contains the omega-3 and omega-6 double bonds. Consequently, omega-3 and omega-6 acids are two separate families of fatty acids since they are not interconvertible in the human body.

Over the past twenty years, health experts have recommended diets lower in saturated fats and higher in polyunsaturated fats. While this advice has been followed by a number of consumers, the incidence of heart disease, cancer, diabetes and many other debilitating diseases has continued to increase steadily. Scientists agree that the type and source of polyunsaturated fats is as critical as the total quantity of fats. The most common polyunsaturated fats are derived from vegetable matter and are lacking in long chain fatty acids (most particularly omega-3 LC-PUFAs). In addition, the hydrogenation of polyunsaturated fats to create synthetic fats has contributed to the rise of certain health disorders and exacerbated the deficiency in some essential fatty acids. Indeed, many medical conditions have been identified as benefiting from omega-3 supplementation. These include acne, allergies, Alzheimer's, arthritis, atherosclerosis, breast cysts, cancer, cystic fibrosis, diabetes, eczema, hypertension, hyperactivity, intestinal disorders, kidney dysfunction, leukemia, and multiple sclerosis. Of note, the World Health Organization has recommended that infant formulas be enriched with omega-3 and omega-6 fatty acids.

The conventionally used polyunsaturates are those derived from vegetable oils, which contain significant amounts of omega-6 (i.e., 18:2n-6) but little or no omega-3. While omega-6 and omega-3 fatty acids are both necessary for good health, it is recommended that they be consumed in a balance of about 4:1. Principal sources of omega-3 are flaxseed oil, fish oils and algal oils. The past decade has seen rapid growth in the production of flaxseed and fish oils. Both types of oil are considered good dietary sources of omega-3 polyunsaturated fats. Flaxseed oil contains no EPA, DHA, DPA or ARA but rather contains alpha-linolenic acid (18:3n-3), a building block enabling the body to manufacture DPA n-3, EPA and DHA. There is evidence however that the rate of metabolic conversion can be slow and unsteady, particularly among those with impaired health. Fish oils vary considerably in the type and level of fatty acid composition depending on the particular species and their diets. For example, fish raised by aquaculture tend to have a lower level of omega-3 fatty acids than those in the wild. Furthermore, fish oils carry the risk of containing environmental contaminants commonly found in fish. In light of the health benefits of such omega-3 and omega-6 LC-PUFAs (chain length greater than 20), it would be desirable to supplement foods with such fatty acids.

Liquid oils such as fish oils and certain microbial oils are known to contain a high content of LC-PUFAs. However, due to their polyunsaturated nature, these oils are not solid at room temperature (i.e., 20° C.), and instead, are in an oil or liquid form. However, solid forms of PUFA-rich oils are desirable for use in certain food applications where liquid oils are not applicable. To form a solid composition, a number of approaches have been tried. A common process used to solidify unsaturated oils consists of partial or full hydrogenation of such oils, so as to obtain semi-solid oils. The partial hydrogenation process results in the formation of “trans”-fatty acids, which have been shown to possess several adverse effects. Hence, by solidifying unsaturated oils using a hydrogenation process, the beneficial properties of the unsaturated oils are substituted by the highly undesirable adverse properties such as the formation of “trans”-fatty acids.

Other methods include mixing the unsaturated oils with “hard” or saturated fats so that the mixture is a semi-solid oil. U.S. Patent Application Publication No. 2007/0003686, the contents of which is incorporated by reference herein in its entirety, discloses a solid fat composition that includes an oil having saturated fat and a microbial oil having a long chain polyunsaturated fatty acid and an emulsifier. Other methods for forming a spreadable, semi-solid fat composition comprising high levels of polyunsaturated fats include using high levels of particular types of emulsifiers, or other thickeners such as fatty alcohols.

### SUMMARY OF THE INVENTION

Until the present invention, there was lacking in the art compositions comprising a solid or semi-solid fat or food product containing high levels of PUFAs, but without exogenously added emulsifiers and/or other types of thickeners. Such compositions and methods to form such compositions would be highly desirable. It would be further desirable to provide a low cost method for making such a composition, said method involving the use of non-hazardous materials, minimal processing steps, and minimal raw material inventory.

The present invention provides methods for producing a solid fat composition comprising: a) mixing an oil comprising saturated fat with an oil comprising at least one LC-PUFA to form a mixture; and b) solidifying the mixture to form a solid fat composition, wherein no exogenous emulsifier is added in producing said solid fat composition.

In some embodiments of the present invention, the oil comprising saturated fat is selected from the group consisting of microbial stearin, unfractionated palm oil, palm olein, palm stearin, palm mid fraction, unfractionated palm kernel oil, palm kernel olein, palm kernel stearin, unfractionated cotton seed oil, cotton seed olein, cotton seed stearin, coconut oil, unfractionated shea butter oil, shea butter stearin, interesterified palm oil blend, interesterified cotton seed oil blend, fish oil stearin, and combinations thereof.

The oil comprising at least one LC-PUFA, which can preferably be a microbial oil, suitable for use in the present invention may be unwinterized. In some embodiments of the present invention, the oil comprises saturated fat. The oil can comprise, but is not limited to, between about 5% to about 70% by weight of at least one LC-PUFA selected from the group consisting of docosahexaenoic acid, omega-3 or omega-6 docosapentaenoic acid, arachidonic acid, and eicosapentaenoic acid.

In some embodiments of the present invention, the oil comprising saturated fat and the oil comprising at least one LC-PUFA are not heated prior to mixing.

The solid fat composition produced by the methods of the present invention can be, or can be incorporated in, without limitation, a food product, a nutritional product and/or a pharmaceutical product. In some embodiments of the present invention, the ratio of the oil comprising at least one LC-PUFA to the oil comprising saturated fat is from about 1:9 to about 9:1 by weight.

The methods of producing a solid fat composition according to the present invention can further comprise deodorizing the mixture of the oil comprising saturated fat and the oil comprising at least one LC-PUFA. In some embodiments of the present invention, the methods of producing a solid fat composition further comprise interesterifying the mixture.

The present invention also provides a solid fat composition comprising a mixture of an oil comprising saturated fat and an oil comprising at least one LC-PUFA, wherein the mixture is a solid composition at room temperature, and wherein the mixture contains no exogenous emulsifier.

In some embodiments of the present invention, the oil comprising saturated fat in the solid fat composition is selected from the group consisting of microbial stearin, unfractionated palm oil, palm olein, palm stearin, palm mid fraction, unfractionated palm kernel oil, palm kernel olein, palm kernel stearin, unfractionated cotton seed oil, cotton seed olein, cotton seed stearin, coconut oil, unfractionated shea butter oil, shea butter stearin, interesterified palm oil blend, interesterified cotton seed oil blend, fish oil stearin, and combinations thereof.

In some embodiments of the present invention, the oil comprising at least one LC-PUFA in the solid fat composition is unwinterized. The oil can comprise saturated fat. In some embodiments of the present invention, the solid fat compositions have an oil that comprises between about 5% to about 70% by weight of at least one LC-PUFA selected from the group consisting of docosahexaenoic acid, omega-3 or omega-6 docosapentaenoic acid, arachidonic acid, and eicosapentaenoic acid.

Preferably, the solid fat compositions of present invention are free of trans-fatty acids. In some embodiments of the present invention, the solid fat compositions have a ratio of oil comprising at least one LC-PUFA to oil comprising saturated fat of from about 1:9 to about 9:1 by weight. The solid fat compositions of the present invention can be, but are not limited to, a food product, a nutritional product, or a pharmaceutical product.

The present invention also provides methods for producing a solid fat composition comprising: a) mixing a stearin comprising at least one LC-PUFA with a second oil comprising saturated fat to form a mixture; and b) solidifying the mixture to form a solid fat composition.

In some embodiments of the present invention, no exogenous emulsifier is added in producing the solid fat compositions.

The stearin suitable for use in the present invention can include, but is not limited to, microbial stearin, fish oil stearin, palm stearin, palm kernel stearin, cotton seed stearin, shea butter stearin, and combinations thereof. In some embodiments of the present invention, the second oil comprising saturated fat is selected from the group consisting of unfractionated palm oil, palm olein, unfractionated palm kernel oil, palm kernel olein, palm mid fraction, coconut oil, unfractionated shea butter oil, unfractionated cotton seed oil, cotton seed olein, interesterified palm oil blend, interesterified cotton seed oil blend, and combinations thereof.

The present invention further provides a solid fat composition comprising a mixture of a stearin composition comprising at least one LC-PUFA and a second oil comprising saturated fat, wherein the composition is solid at room temperature. In some embodiments of the present invention, the stearin is selected from the group consisting of microbial stearin, fish oil stearin, palm stearin, palm kernel stearin, cotton seed stearin, shea butter stearin, and combinations thereof. The second oil comprising saturated fat suitable for use in the present invention can include, but is not limited to, unfractionated palm oil, palm olein, unfractionated palm kernel oil, palm kernel olein, palm mid fraction, coconut oil, unfractionated shea butter oil, shea butter stearin, unfractionated cotton seed oil, cotton seed olein, interesterified palm oil blend, interesterified cotton seed oil blend, and combinations thereof.

### BRIEF DESCRIPTION OF THE DRAWINGS

FIG. 1 illustrates various alternative embodiments for producing oils comprising saturated fat and oils comprising at least one LC-PUFA suitable for use in the present invention.

FIG. 2 illustrates various alternative embodiments for producing minimally processed PUFA oils suitable for use in the present invention.

FIG. 3 illustrates various alternative embodiments for producing a PUFA-containing solid fat composition of the present invention.

### DETAILED DESCRIPTION OF THE INVENTION

The food, nutritional, and pharmaceutical product compositions and methods for preparation of the same, as taught by the present invention, allow for increased intake of nutrients, particularly LC-PUFAs, and more particularly omega-3 and omega-6 LC-PUFAs, which can provide health benefits to those consuming such products. The present invention provides high-quality PUFA-containing solid fat products and uses and production thereof. In some embodiments of the present invention, a PUFA-containing solid fat product comprises a high-quality PUFA-containing oil product prepared with minimal processing and has improved functionality, improved stability and is compatible with a broad range of applications including the natural and/or organic market sector. For example, the solid fat compositions of the present invention comprising LC-PUFAs can be used in, or as, nutritional products, food products, and/or pharmaceutical products (medicinal and/or therapeutic). In some embodiments of the present invention, the oils for making products of the invention are microbial oils containing LC-PUFAs derived from a microbial biomass.

In some embodiments of the present invention, the oil comprising at least one LC-PUFA can be a minimally processed microbial oil that is a high-quality PUFA-containing oil product that can be used as a starting material for producing the solid fat compositions of the present invention. The process for producing such minimally processed microbial oils includes extracting an oil-containing fraction comprising at least one LC-PUFA from a microbial biomass to produce a microbial oil. Microbial sources and methods for growing microorganisms comprising nutrients and/or LC-PUFAs for recovery in microbial oils are known in the art (Industrial Microbiology and Biotechnology, 2^{nd} edition, 1999, American Society for Microbiology). Preferably, the microorganisms are cultured in a fermentation medium in a fermentor. The methods and compositions of the present invention are applicable to any industrial microorganism that produces LC-PUFA.

Microbial sources can include a microorganism such as an algae, bacteria, fungi (including yeast) and/or protist. Preferred organisms include those selected from the group consisting of golden algae (such as microorganisms of the kingdom Stramenopiles), green algae, diatoms, dinoflagellates (such as microorganisms of the order Dinophyceae including members of the genus Crypthecodinium such as, for example, Crypthecodinium cohni), yeast, and fungi of the genera Mucor and Mortierella, including but not limited to Mortierella alpina and Mortierella sect. schmuckeri. Members of the microbial group Stramenopiles include microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). The Labrinthulids include the genera Labyrinthula (species include algeriensis, coenocystis, chattonii, macrocystis, macrocystis atlantica, macrocystis macrocystis, marina, minuta, roscofensis, valkanovii, vitellina, vitellina pacifica, vitellina vitellina, zopfi), Labyrinthomyxa (species include marina), Labyrinthuloides (species include haliotidis, yorkensis), Diplophrys (species include archenri), Pyrrhosorus* (species include marinus), Sorodiplophrys* (species include stercorea), Chlamydomyxa* (species include labyrinthuloides, montana). (*=there is no current general consensus on the exact taxonomic placement of these genera). While a wide variety of microorganisms can be suitable sources of material for the present invention, for the sake of brevity, convenience and illustration, this detailed description of the invention will discuss processes for growing microorganisms which are capable of producing lipids comprising omega-3 and/or omega-6 polyunsaturated fatty acids, in particular microorganisms that are capable of producing DHA, DPA n-3, DPA n-6, EPA or ARA. Additional preferred microorganisms are algae, such as Thraustochytrids of the order Thraustochytriales, including Thraustochyrrium (including Ulkenia) and Schizochytrium, and including Thraustochytriales which are disclosed in commonly assigned U.S. Pat. Nos. 5,340,594 and 5,340,742, both issued to Barclay, which are incorporated herein by reference in their entirety. More preferably, the microorganisms are selected from the group consisting of microorganisms having the identifying characteristics of ATCC number 20888, ATCC number 20889, ATCC number 20890, ATCC number 20891 and ATCC number 20892. Since there is some disagreement among experts as to whether Ulkenia is a separate genus from the genus Thraustochytrium, for the purposes of this application, the genus Thraustochytrium will include Ulkenia. Also preferred are strains of Mortierella sect. schmuckeri (e.g., including microorganisms having the identifying characteristics of ATCC 74371) and Mortierella alpina (e.g., including microorganisms having the identifying characteristics of ATCC 42430). Also preferred are strains of Crypthecodinium cohnii, including microorganisms having the identifying characteristics of ATCC Nos. 30021, 30334-30348, 30541-30543, 30555-30557, 30571, 30572, 30772-30775, 30812, 40750, 50050-50060, and 50297-50300. Also preferred are mutant strains derived from any of the foregoing, and mixtures thereof. Oleaginous microorganisms are also preferred. As used herein, “oleaginous microorganisms” are defined as microorganisms capable of accumulating greater than 20% of the weight of their cells in the form of lipids. Genetically modified microorganisms that produce LC-PUFAs are also suitable for the present invention. These can include naturally LC-PUFA-producing microorganisms that have been genetically modified as well as microorganisms that do not naturally produce LC-PUFAs but that have been genetically engineered to do so.

Suitable organisms may be obtained from a number of available sources, including by collection from the natural environment. The American Type Culture Collection currently lists many publicly available strains of the microorganisms identified above. As used herein, any microorganism, or any specific type of organism, includes wild strains, mutants, or recombinant types. Growth conditions in which to culture these organisms are known in the art, and appropriate growth conditions for at least some of these organisms are disclosed in, for example, U.S. Pat. No. 5,130,242, U.S. Pat. No. 5,407,957, U.S. Pat. No. 5,397,591, U.S. Pat. No. 5,492,938, U.S. Pat. No. 5,711,983, U.S. Pat. No. 5,882,703, U.S. Pat. No. 6,245,365, and U.S. Pat. No. 6,607,900, all of which are incorporated herein by reference in their entirety.

Microbial oils useful in the present invention can be recovered from microbial sources by any suitable means known to those in the art. For example, the oils can be recovered by extraction with solvents such as chloroform, hexane, methylene chloride, methanol and the like, or by supercritical fluid extraction. Alternatively, the oils can be extracted using extraction techniques, such as are described in U.S. Pat. No. 6,750,048 and PCT Patent Application Serial No. US01/01806, both filed Jan. 19, 2001, and entitled “Solventless Extraction Process,” both of which are incorporated herein by reference in their entirety. Additional extraction and/or purification techniques are taught in PCT Patent Application Serial No. PCT/IB01/00841 entitled “Method for the Fractionation of Oil and Polar Lipid-Containing Native Raw Materials” filed Apr. 12, 2001; PCT Patent Application Serial No. PCT/IB01/00963 entitled “Method for the Fractionation of Oil and Polar Lipid-Containing Native Raw Materials Using Water-Soluble Organic Solvent and Centrifugation” filed Apr. 12, 2001; U.S. Provisional Patent Application Ser. No. 60/291,484 entitled “Production and Use of a Polar Lipid-Rich Fraction Containing Stearidonic Acid and Gamma Linolenic Acid from Plant Seeds and Microbes” filed May 14, 2001; U.S. Provisional Patent Application Ser. No. 60/290,899 entitled “Production and Use of a Polar-Lipid Fraction Containing Omega-3 and/or Omega-6 Highly Unsaturated Fatty Acids from Microbes, Genetically Modified Plant Seeds and Marine Organisms” filed May 14, 2001; U.S. Pat. No. 6,399,803 entitled “Process for Separating a Triglyceride Comprising a Docosahexaenoic Acid Residue from a Mixture of Triglycerides” issued Jun. 4, 2002 and filed Feb. 17, 2000; and PCT Patent Application Serial No. US01/01010 entitled “Process for Making an Enriched Mixture of Polyunsaturated Fatty Acid Esters” filed Jan. 11, 2001; all of which are incorporated herein by reference in their entirety. The extracted oils can be evaporated under reduced pressure to produce a sample of concentrated oil material. Processes for the enzyme treatment of biomass for the recovery of lipids are disclosed in U.S. Provisional Patent Application No. 60/377,550, entitled “HIGH-QUALITY LIPIDS AND METHODS FOR PRODUCING BY ENZYMATIC LIBERATION FROM BIOMASS,” filed on May 3, 2002; PCT Patent Application Serial No. PCT/US03/14177 entitled “HIGH-QUALITY LIPIDS AND METHODS FOR PRODUCING BY ENZYMATIC LIBERATION FROM BIOMASS,” filed on May 5, 2003; copending U.S. patent application Ser. No. 10/971,723, entitled “HIGH-QUALITY LIPIDS AND METHODS FOR PRODUCING BY LIBERATION FROM BIOMASS,” filed on Oct. 22, 2004; EP Patent Publication 0 776 356 and U.S. Pat. No. 5,928,696, both entitled “Process for extracting native products which are not water-soluble from native substance mixtures by centrifugal force,” all of which are incorporated herein by reference in their entirety.

In preferred embodiments, the microbial oils suitable for use in the present invention are high quality microbial crude oils prepared by processes as described above. Such oils have significant advantages over, for example, fish oils that typically provide poor quality crude oils because recovery from fish biomass typically involves cooking and hexane extraction and because the oil can contain contaminants, other undesirable components and/or undesirable fatty acid profiles.

The oil comprising at least one LC-PUFA includes at least one LC-PUFA. Preferred PUFAs of the present invention include C20, C22, or C24 omega-3 or omega-6 PUFAs.

Preferably, the PUFA is a long chain PUFA (LC-PUFA), comprising a C20 or C22 omega-3, or a C20 or C22 omega-6 PUFA. An LC-PUFA of the present invention contains preferably at least two double bonds, more preferably at least three double bonds, and even more preferably at least four double bonds. PUFAs having 4 or more unsaturated carbon-carbon bonds are also commonly referred to as highly unsaturated fatty acids, or HUFAs. In particular, the LC-PUFA can include: docosahexaenoic acid (at least about 10, about 20, about 30, about 35, about 40, about 50, about 60, about 70 or about 80 weight percent of total fatty acids), docosapentaenoic acid n-3 (at least about 10, about 20, about 30, about 40, about 50, about 60, about 70 or about 80 weight percent of total fatty acids), docosapentaenoic acid n-6 (at least about 10, about 20, about 30, about 40, about 50, about 60, about 70 or about 80 weight percent of total fatty acids), arachidonic acid (at least about 10, about 20, about 30, about 40, about 50, about 60, about 70 or about 80 weight percent of total fatty acids) and/or eicosapentaenoic acid (at least about 10, about 20, about 30, about 40, about 50, about 60, about 70 or about 80 weight percent of total fatty acids). The PUFAs can be in any of the common forms found in natural lipids including but not limited to triacylglycerols, diacylglycerols, monoacylglycerols, phospholipids, free fatty acids, esterified fatty acids, or in natural or synthetic derivative forms of these fatty acids (e.g. calcium salts of fatty acids, ethyl esters, etc). In preferred embodiments, the microbial oil-containing fraction comprises at least about 70 wt. %, at least about 80 wt. %, at least about 90 wt. %, or at least about 95 wt. % of the PUFAs in the fraction in the triglyceride form. The term LC-PUFA, as used in the present invention, can refer to either an oil comprising a single omega-3 LC-PUFA (such as DHA), an oil comprising a single omega-6 LC-PUFA (such as ARA or DPA n-6), or an oil comprising a mixture of two or more LC-PUFAs (such as DHA, DPA n-6, ARA, and EPA). In preferred embodiments, the product comprises an LC-PUFA in combination with at least one other nutrient.

In addition to the use of a microbial biomass for the extraction of oils containing LC-PUFAs, plant-based sources, such as oil seeds can also be used as a biomass for extraction or recovery of LC-PUFAs including, for example, plants from any higher plant, and particularly consumable plants, including crop plants and especially plants used for their oils. Such oils extracted from a plant biomass can be processed and treated as disclosed herein to produce oil products. Such plants can include, for example: canola, soybeans, rapeseed, linseed, corn, safflowers, sunflowers and tobacco. Other preferred plants include those plants that are known to produce compounds used as pharmaceutical agents, flavoring agents, nutraceutical agents, functional food ingredients or cosmetically active agents or plants that are genetically engineered to produce these compounds/agents. PUFA-producing plants include those genetically engineered to express genes that produce PUFAs and those that produce PUFAs naturally. Such genes can include genes encoding proteins involved in the classical fatty acid synthase pathways, or genes encoding proteins involved in the PUFA polyketide synthase (PKS) pathway. The genes and proteins involved in the classical fatty acid synthase pathways, and genetically modified organisms, such as plants, transformed with such genes, are described, for example, in: Napier and Sayanova, Proceedings of the Nutrition Society (2005), 64:387-393; Robert et al., Functional Plant Biology (2005) 32:473-479; or U.S. Patent Application Publication 2004/0172682. The PUFA PKS pathway, genes and proteins included in this pathway, and genetically modified microorganisms and plants transformed with such genes for the expression and production of PUFAs, are described in detail in: U.S. Pat. No. 6,140,486; U.S. Pat. No. 6,566,583; U.S. Patent Application Publication No. 20020194641; U.S. Pat. No. 7,211,418; U.S. Patent Application Publication No. 20050100995A1; U.S. Patent Application Publication No. 20070089199; PCT Publication No. WO 05/097982; and U.S. Patent Application Publication No. 20050014231; each of which is incorporated herein by reference in its entirety.

Such plants, and particularly oil seeds, can be treated by conventional methods to recover oils, such as by cleaning, dehulling and grinding. The seeds can then be pressed to produce an oil or contacted with a solvent, such as after flaking, to extract an oil. Suitable solvents can include organic solvents, water miscible solvents and water. A preferred solvent is hexane.

Another biomass source of PUFA-containing oils suitable for the compositions and methods of the present invention includes an animal source. Examples of animal sources include aquatic animals (e.g., fish, marine mammals, and crustaceans such as krill and other euphausids) and animal tissues (e.g., brain, liver, eyes, etc.) and animal products such as eggs or milk. Techniques for recovery of PUFA-containing oils from such sources are known in the art.

In some embodiments of the present invention, the oil (such as a microbial oil) that is used to produce a solid fat composition has been subjected to a treatment such as refining, bleaching, deodorization, winterization, or chill filtration, or to a combination of these treatments.

In some embodiments of the present invention, a further characteristic of PUFA-containing oil products useful is that they contain saturated fatty acids that are at least sufficient to visually affect the oil-containing fraction. Many PUFA-containing oil products contain sufficient amounts of saturated fatty acids in forms that, at room temperature (i.e., 20° C.), visually affect the oil, such as by causing cloudiness in the oil. Some such products are even paste-like due to the presence of saturated fatty acids, for example, because they contain sufficient saturated fatty acids in the form of triglycerides. While in conventional processing, such oil products are winterized to remove the saturated fatty acids, such oil products may be used in the present invention without winterization, as discussed in more detail below.

In preferred embodiments of the present invention, oils useful in the present invention have a lipid profile particularly suitable for producing solid or semi-solid compositions comprising LC-PUFAs. More particularly, such oils are relatively concentrated in highly unsaturated compounds (e.g., 4, 5 or higher points of unsaturation), relatively concentrated in saturated compounds, and/or relatively unconcentrated in mono-, di-, and tri-saturated compounds. Such compositions can be characterized as having a bimodal distribution of compounds in terms of saturation, i.e., high amounts of saturated compounds and high amounts of highly unsaturated compounds, with low amounts of compounds with intermediate amounts of unsaturatation. For example, such oils can have greater than about 20% by weight, greater than about 25% by weight, greater than about 30% by weight, greater than about 35% by weight, greater than about 40% by weight, greater than about 45% by weight, or greater than about 50% by weight of highly unsaturated compounds having 4 or more points of unsaturation. In other embodiments, such oils can have greater than about 20% by weight, greater than about 25% by weight, greater than about 30% by weight, greater than about 35% by weight, greater than about 40% by weight, greater than about 45% by weight, or greater than about 50% by weight of highly unsaturated compounds having 5 or more points of unsaturation. Alternatively, or in addition, such oils can have greater than about 30% by weight, greater than about 35% by weight, greater than about 40% by weight, greater than about 45% by weight, or greater than about 50% by weight of saturated compounds. Alternatively, or in addition, such oils can have less than about 25% by weight, less than about 20% by weight, less than about 15% by weight, less than about 10% by weight, or less than about 5% by weight of mono-, di- or tri-saturated compounds.

Production of minimally processed high-quality PUFA-containing oil products comprising at least one LC-PUFA can further include treating the extracted oil-containing fraction produced as described herein, such as those oil-containing fractions described in U.S. Patent Publication No. US-2007-003686-A1. Such further treatment can include, without limitation, a process of vacuum evaporation to produce an oil product comprising at least one LC-PUFA.

The process of vacuum evaporation can include desolventization and/or drying by high vacuum evaporation, and is generally known in the art. This process includes subjecting an extracted oil to vacuum conditions, preferably at high temperatures (e.g., from about 50° C. to about 70° C.). For example, the oil can be subjected to a vacuum of greater than a vacuum of about 100 mm Hg, greater than a vacuum of about 70 mm Hg, and greater than a vacuum of about 50 mm Hg. As used herein, for example, reference to “a vacuum of greater than a vacuum of about 100 mm Hg” means a stronger vacuum such as, e.g., a vacuum of 90 mm Hg or 80 mm Hg. Under these conditions, any solvents, water or other components in the extracted oil having a boiling point below the oil will be driven off.

The process of deodorization is generally known in the art and includes subjecting an extracted oil to vacuum conditions to remove any low molecular weight components that may be present. Typically, these components are removed by sparging with steam at high temperatures, under high vacuum. For example, the oil is generally subjected to a vacuum greater than those noted above for desolventization. Specifically, the vacuum can be a vacuum of greater than a vacuum of about 50 mm Hg, greater than a vacuum of about 25 mm Hg, greater than a vacuum of about 12 mm Hg, greater than a vacuum of about 6 mm Hg, and typically can be between a vacuum of about 12 mm Hg and a vacuum of about 6 mm Hg or between a vacuum of about 6 mm Hg and a vacuum of about 1 mm Hg. This process also destroys many peroxide bonds that may be present and reduces, or removes off, odors and helps improve the stability of the oil. In addition, under these conditions, solvents, water and/or other components in the extracted oil having a boiling point below the oil will be driven off. Deodorization is typically performed at high temperatures, such as temperatures between about 190° C. and about 220° C.

In some embodiments of the present invention, the PUFA-containing oil that is used in the present invention is suitable for consumption by humans and non-human animals. That is, the organoleptic properties of the oil are such that consumption of the product is acceptable to humans and non-human animals. Specifically, the oil can contain low concentrations of free fatty acids, phosphorous, peroxide values, anisidine values, soaps and heavy metals. Production of this oil by the methods described above minimizes the amount of downstream processing required to bring the oil to acceptable commercial conditions. Specific modifications that may be incorporated into the production of a PUFA-containing oil suitable for use in the present invention include the elimination of a solvent winterization step, the elimination of a caustic refining process, the elimination of a chill filtration process, and the possible elimination of a bleaching process. In addition, a high-vacuum evaporation process can be substituted for a deodorization process. The foregoing process description facilitates the production of a solid or semi-solid product by retaining the presence of sufficient saturated compounds to prevent the composition from being liquid at room temperature (i.e., about 20° C.). The foregoing process allows production of edible oils from crude oils, and particularly crude microbial oils, with exceptionally high recoveries (95-100%) that are compatible with the natural and/or organic market sector.

In various embodiments, oil products suitable for use in the present invention, such as oils produced without being subjected to one or more of the conventional processing steps of solvent winterization, caustic refining process, chill filtration process, and/or a bleaching process, have low concentrations of free fatty acids. Measurement of concentrations of free fatty acids of oils is well known in the art. More particularly, oils suitable for use in the present invention can have a free fatty acid content of less than about 0.5 wt. %, less than about 0.1 wt. %, and less than about 0.05 wt. %.

In various embodiments, oil products suitable for use in the present invention, such as oils produced without being subjected to one or more of the conventional processing steps of solvent winterization, caustic refining process, chill filtration process, and a bleaching process, have low phosphorous values. Measurement of phosphorous values of oils is well known in the art. More particularly, oils suitable for use in the present invention can have a phosphorous value of less than about 10 ppm, less than about 5 ppm, and about 0 ppm.

In various embodiments, oil products suitable for use in the present invention, such as oils produced without being subjected to one or more of the conventional processing steps of solvent winterization, caustic refining process, chill filtration process, and a bleaching process, have low peroxide values. Measurement of peroxide values of oils is well known in the art. More particularly, oils suitable for use in the present invention can have a peroxide value of less than about 2 meq/kg, less than about 1 meq/kg, and about 0 nm eq/kg.

In various embodiments, oil products suitable for use in the present invention, such as oils produced without being subjected to one or more of the conventional processing steps of solvent winterization, caustic refining process, chill filtration process, and a bleaching process, have low anisidine values. Measurement of anisidine values of oils is well known in the art. More particularly, oils suitable for use in the present invention can have an anisidine value of less than about 5, less than about 3, less than about 2, less than about 1, less than about 0.5, less than about 0.3, less than about 0.1, and below detection.

In various embodiments, oil products suitable for use in the present invention, such as oils produced without being subjected to one or more of the conventional processing steps of solvent winterization, caustic refining process, chill filtration process, and a bleaching process, have low concentrations of soaps. Measurement of concentrations of soap of oils is well known in the art. More particularly, oils suitable for use in the present invention can have soap contents of less than about 5 wt. %, less than about 2.5 wt. %, and of 0 wt. %.

In various embodiments, oil products suitable for use in the present invention, such as oils produced without being subjected to one or more of the conventional processing steps of solvent winterization, caustic refining process, chill filtration process, and a bleaching process, have low heavy metal values. Measurement of heavy metal values of oils is well known in the art. More particularly, oils suitable for use in the present invention can have Fe concentrations of less than about 1 ppm, less than about 0.5 ppm, and preferably at about 0 ppm; Pb concentrations of less than about 1 ppm, less than about 0.2 ppm, and preferably at about 0 ppm; Hg concentrations of less than about 0.1 ppm, less than about 0.04 ppm, and preferably at about 0 ppm; Ni concentrations of less than about 0.1 ppm, less than about 0.01 ppm, and preferably at about 0 ppm; and Cu concentrations of less than about 1 ppm, less than about 0.2 ppm, and preferably at about 0 ppm.

Processes to produce minimally processed high-quality PUFA-containing oil products having at least one LC-PUFA can optionally include a step of bleaching the oil either before or after the step of deodorization or the step of high vacuum fractionation, although it is more commonly conducted before the step of deodorization. Bleaching of oils is well known in the art and can be accomplished in conventional processes. Specifically, for example, a silica adsorbent (such as, Trysil 600 (Grace Chemicals)) for removing remnant soap and a bleaching clay can be introduced to the oil and then filtered out. Typically, the silica adsorbent is added before the bleaching clay.

Processes to produce high-quality PUFA-containing oil products having at least one LC-PUFA can include a process to produce a liquid LC-PUFA-containing oil fraction and an LC-PUFA-containing solid fat product. Such a process includes a step of fractionating a high quality crude oil, and preferably a microbial crude oil, as disclosed herein, into an oil product and related solid fat product. Such crude oil products can be prepared by extracting an oil-containing fraction containing at least one LC-PUFA and saturated fatty acids from a biomass, and in some embodiments from a microbial biomass. The oil-containing fraction can be treated by winterization, chill filtration, vacuum evaporation and/or other means to produce a liquid oil product comprising at least one LC-PUFA and a solid product comprising at least one LC-PUFA. Such other means can include filtration to separate the liquid oil fraction from a solid composition.

The solid fraction components, which may include adsorbents, can be recovered by solid/liquid separation techniques. Any adsorbents can be separated from the solid fraction by heating the adsorbents and solid fat material to melt the solid fat material. The adsorbents can then be separated from the melted solids, by filtering, for example, and the melted solids can then be resolidified by cooling.

The recovered solid fraction will contain a high level of LC-PUFA. In preferred embodiments, the solid fraction will comprise at least about 20%, at least about 25%, at least about 30% by weight LC-PUFA and, in some embodiments, DHA. In some embodiments of the present invention, the solid fraction comprises stearin. Each of the clear oil and the solid can be used, for example, as a food, or as a food additive.

Oil products produced in accordance with the present invention can be solid or semi solid materials. As used herein, the term “oil” will include those materials that are solid or semi solid at room temperature, as well as those materials that are liquid at room temperature.

As used herein, the term “semi-solid oil” refers to a semi-solid, fluid and pourable fat product at normal room temperatures.

As used herein, the term “solid” or “plastic” fat product refers to a solid, non-fluid and non-pourable fat product at typical storage temperature of about 25° C.

Processes to produce minimally processed, high-quality PUFA-containing oil products having at least one LC-PUFA can optionally include a step of fractionating the oil into an olein fraction and a stearin fraction after either the step of deodorization or the step of high vacuum fractionation. Fractionation of oils into olein and stearin fractions can be applied to any crude, or bleached or deodorized oil to produce a clear olein fraction and a hard stearin fraction. Due to differences in their physical properties, olein and stearin can be used in different food applications. In conventional processes, stearin is a byproduct of miscella winterization and chill filtration and is disposed of, resulting in −30% losses. Fractionation therefore allows production of a saleable stearin fraction. An example of this type of fractionation is described below, in Example 4.

The present invention also provides for the recovery of LC-PUFA-containing stearin from the winterization (i.e., chill filtration, miscella winterization, etc.,) of LC-PUFA-containing oils. In some embodiments of the present invention, the LC-PUFA containing stearin is recovered from winterization of an LC-PUFA-containing oil without fractionation of the oil. In some embodiments of the present invention, the LC-PUFA-containing stearin is a microbial stearin. As used herein, “microbial stearin” includes stearin recovered from the fractionation or other processing (such as miscella winterization and chill filtration) of microbial oils.

In some embodiments of the present invention, the LC-PUFA-containing stearin comprises about 15% to about 50% by weight LC-PUFAs. For example, the LC-PUFA-containing stearin of the present invention can comprise at least about 20%, at least about 25%, at least about 30% or at least about 35% by weight LC-PUFA, and in particular DHA. Such LC-PUFA-containing stearin is suitable to produce the solid fat compositions of the present invention.

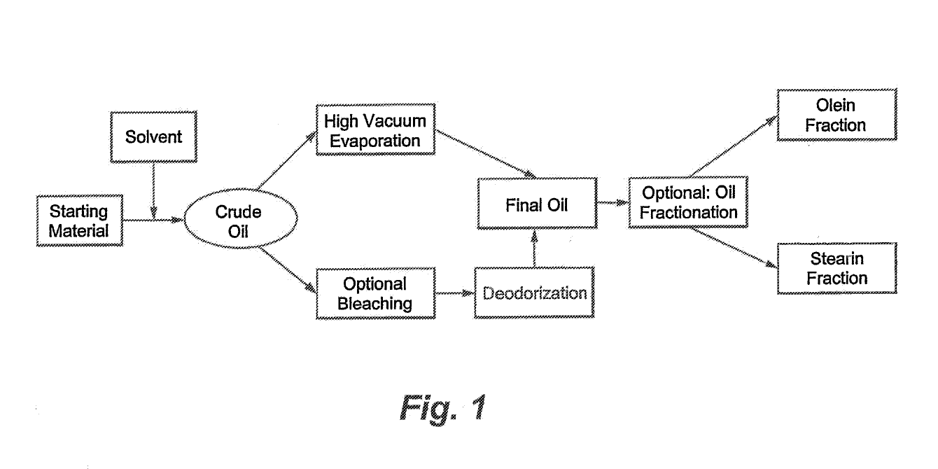

With reference to FIG. 1, various alternative methods of producing suitable oils comprising saturated fat and oils comprising at least one LC-PUFA are illustrated. A starting material, such as a biomass or such as a spray dried biomass, can be subjected to treatment by a solvent for extraction of a crude oil. Such crude oils will include LC-PUFAs. The crude oil can be subjected to high vacuum evaporation which will remove extraction solvents, water and other components in the crude oil having a lower boiling point than the desired oil components. Alternatively, the crude oil can be subjected to an optional bleaching step, such as to remove carotenoids. The optionally treated crude oil is then subjected to deodorization by sparging the oil with steam at high temperatures, under high vacuum. The final oil product produced by either the high vacuum evaporation or the deodorization can then be optionally treated by fractionation into an olein fraction and a stearin fraction.

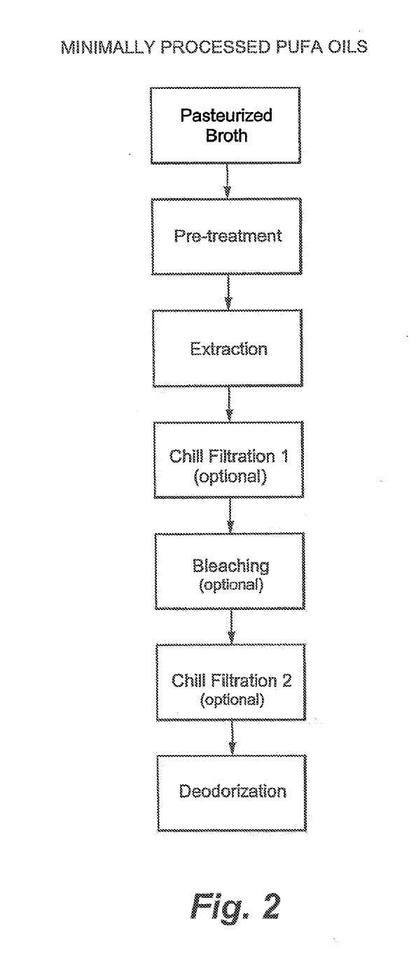

With reference to FIG. 2, various alternative methods of producing minimally processed PUFA oils suitable for use in the present invention are illustrated by a flow sheet. In its most basic form, the process includes the steps of starting with a pasteurized fermentation broth containing a biomass which, in some embodiments, is a microbial biomass. The broth is pretreated to release oil from the cells by lysing, such as by enzymatic treatment or mechanical disruption. The pretreated fermentation broth is then subjected to an extraction step to produce an oil. The process then includes a deodorization step, as described herein. In an alternative embodiment, the process also includes a bleaching step by which the extracted oil is subjected to bleaching prior to the step of deodorization. In further alternative embodiments, winterization steps (i.e., chill filtration) can be conducted on the extracted oil prior to the step of bleaching and/or between the step of bleaching and deodorization.

The processes for producing minimally processed oils, and the resulting products, described herein have a number of significant advantages. Compared to conventional methods of producing PUFA-containing oil products, these processes have a lower cost, reduced processing requirements, increased manufacturing throughput, increased safety of the processing steps, and eliminate waste/byproduct streams. Moreover, the current processes are consistent with the natural and/or organic market sector.

As described more fully below, the high quality PUFA-containing solid fat products of the present invention can be used in a variety of food products and applications. The solid fat products can be consumed directly by humans as a nutritional, dietary, medicinal, or pharmaceutical product. In addition, the solid fat products can be combined with any known human food or liquid for consumption by humans to improve nutrition. The solid fat products can also be fed to animals directly as a feed or as a supplement to animal feed. In this manner, any animal-based food products can have enhanced quality when consumed by humans. The use of the solid fat products of the present invention can also be extended to liposomes, drug carriers, cosmetics, pet food, and aquaculture feeds.

In some embodiments of the present invention, the oil products described herein can be combined to produce a blend. For example, a minimally processed oil from Crypthecodinium cohnii can be blended with a physically refined oil from Mortierella alpina and the resulting blend can be used in the production of the solid fat compositions of the present invention. As another example, blends of ARA-containing oils and DHA-containing oils using oils as described herein can be produced in a variety of different ratios of ARA to DHA. Such blends can include ratios of ARA:DHA from about 1:1 to about 2:1. More particularly, the blends can be produced having ARA:DHA ratios of about 1:1, 1.25:1, 1.5:1, 1.75:1 or 2:1.

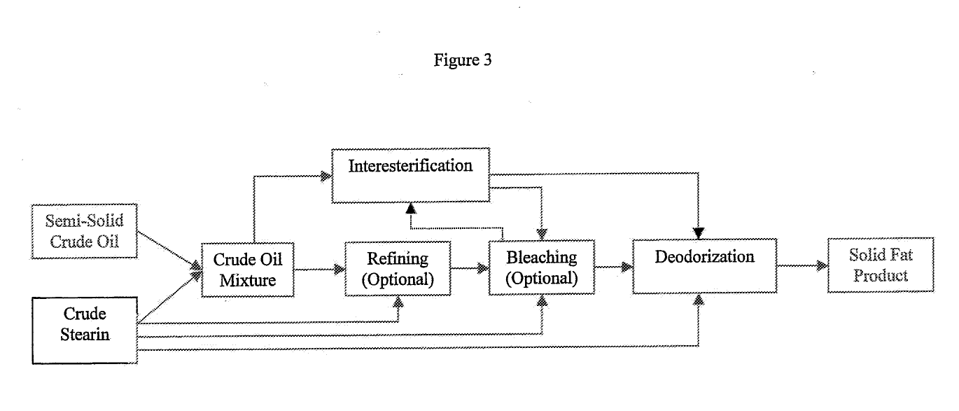

With reference to FIG. 3, various alternative embodiments of the present invention for producing a solid fat composition are illustrated. In one embodiment, a semi-solid crude oil can be combined with a crude stearin to form a mixture. This mixture is then deodorized prior to being formed into a solid fat product. The process of forming a solid fat product may optionally include a step of refining, a step of bleaching and/or a step of interesterification. In another embodiment, the crude stearin can be deodorized and formed into a solid fat product. The process of forming a solid fat product from stearin alone can optionally include a step of refining and/or a step of bleaching.

In some embodiments of the present invention, the methods for producing a solid fat composition further include a step of interesterifying the mixture of an oil comprising saturated fat and an oil comprising at least one LC-PUFA. Such interesterification reactions can also be carried out for mixtures of stearin and an oil comprising saturated fat. Methods of performing such interesterification include treating the mixture with a chemical catalyst or with enzymes.

Typically, chemical interesterification can be carried out using sodium methoxide or sodium ethoxide or an alkali metal as a catalyst. In some embodiments, about 0.05% to about 1.5% by weight of sodium methoxide or sodium ethoxide can be used in the interesterification process. In some embodiments, about 0.1% to about 10% by weight of an alkali metal can be used in the interesterification process. In some embodiments, about 0.05% to about 1.0% by weight of sodium potassium alloy can be used in the interesterification process. In preferred embodiments, the oil mixture is dried under vacuum of between 5 mmHg to 15 mmHg at a temperature of between 90° C. to 120° C. for 0.5 to 2 hours prior to chemical interesterification. In some embodiments, the interesterification reaction can be carried out at a temperature of about 60° C. to about 105° C. for a time period ranging from about 0.5 hours to about 2 hours.

Enzymatic interesterification can be performed with a variety of enzymes, including lipases. Lipases can be of plant or microbial origin, and can be sn-1,3 specific or non-specific. In some embodiments, the enzymatic interesterification is carried out at a temperature of between about 45° C. to about 75° C. for a time period ranging from about 0.5 hour to about 24 hours. Microbial lipases suitable for use in the interesterification include lipases from Rhizomucor miehei, Candida antarctica, Aspergillus niger, Pseudomonas cepacia, Pseudomonas fluorescens, Geotrichum candidum, Rhizopus delemar, Rhizopus oryzae, and Thermomyces lanuginosus.

The high quality PUFA-containing oil products described herein can be used as a starting material for the solid fat compositions that are described in detail below. It should be appreciated, however, that the starting material for the solid fat compositions of the present invention is not limited to the use of the minimally processed oil products described herein.

The inventors have surprisingly discovered that in preferred embodiments of the solid fat composition of the present invention, an oil comprising saturated fat and a oil comprising at least one LC-PUFA can be mixed and solidified to form a solid fat composition, without the need for the addition of an emulsifier. As used herein, the term “no exogenous emulsifier” refers to a composition or process in which no emulsifier is added to form a composition of the invention.

In some embodiments of the present invention, an unwinterized form of an LC-PUFA rich oil, including an unwinterized microbially-derived docosahexaenoic acid-containing oil (DHA oil), can be used as a starting material for the solid fat compositions of the present invention. The inventors have surprisingly discovered that in preferred embodiments of the solid fat composition of the present invention, the solid fat composition is stable and remains homogenous without the use of an emulsifier. The processes for making such compositions thereby can avoid the need for hydrogenation of oils, mixing these oils with emulsifiers, or other agents such as thickening-type agents. Typically, refined oils, i.e., liquid fish oils or microbial oils, are produced as an initial crude oil that is then subjected to refining (which removes phospholipids and free fatty acids) and bleaching (to remove pigments) steps. The oil is then typically winterized to remove saturated fats. In some embodiments of the present invention, however, winterization is not required prior to using the oil as a starting material in the production of solid fat compositions. In addition, unwinterized oil seed oils, as described above, can be used as an alternative to microbial oils as described below.

In some embodiments of the present invention, the method of producing a solid fat composition includes the step of mixing an oil comprising a saturated fat with an oil comprising at least one LC-PUFA to form a mixture. The mixture is then solidified to form a solid fat composition. In preferred embodiments of the present invention, the mixture and resulting composition contain less than about 0.01% by weight, less than about 0.009% by weight, less than about 0.005% by weight, or less than about 0.002% by weight of an emulsifier. In some embodiments of the present invention, no exogenous emulsifier is added in producing the solid fat compositions.

The elimination of the need for addition of emulsifier in the production of solid fat compositions according to the present invention reduces the cost of production and simplifies the production process. Without being bound by theory, the inventors believe that the use of the proper ratio of the amount of an oil comprising at least one LC-PUFA to the amount of oil comprising saturated fat contributes to the formation of a stable, homogenous solid fat composition where an emulsifier is not used. In some embodiments, the ratio of the amount of an oil comprising at least one LC-PUFA (such as a microbial oil) to the amount of an oil comprising saturated fat (such as stearin) is from about 1:9 to about 9:1 by weight, from about 1:6 to about 6:1 by weight, or from about 1:3 to about 3:1 by weight. In some embodiments of the present invention, the ratio of the amount of an oil comprising at least one LC-PUFA to the amount of an oil comprising saturated fat is about 1:1, about 3:1, or about 6:1 by weight.

The saturated fats present in the unwinterized oil will also give a more solid consistency to the oil (as compared to winterized liquid oil). The methods of the present invention for producing a solid fat composition also overcome the tendency of an unwinterized oil to appear grainy (due to the crystallization of triglycerides) causing such unwinterized oils to appear as a thick liquid oil with particles. Upon standing at room temperature, unwinterized oil separates, giving a product that appears as a thick liquid oil with solids in it. Processes described herein produce a smooth product of uniform appearance that is stable, with no apparent separation, when left standing at room temperature. The resulting product can have the consistency of shortening.

As used herein, a “solid fat composition” refers to a composition that is solid, or semi-solid, at room temperature (i.e., 25° C.). Physicochemical properties of fats and oils include their viscosity and melting temperature. Preferably, a solid fat composition of the present invention will have a melting temperature of at least about 30° C., at least about 35° C., at least about 40° C., and at least about 50° C. Melting temperatures will vary depending on the number of different chemical entities present. Typically, a mixture of several triglycerides has a lower melting point than would be predicted based on the melting points of the individual triglycerides. The mixture will also have a broader melting range than that of its individual components. Monoglycerides and diglycerides have higher melting points than triglycerides of similar fatty acid composition.

In preferred embodiments, the solid fat composition will remain soft enough to spread onto food products. Preferably, at room temperatures, the composition will be viscous, have retarded flow properties, and/or be more adherent to surfaces than the starting materials from which the product is made.

In some embodiments of the present invention, the solid fat compositions have a drop point of between about 20° C. to about 60° C. For example, the solid fat compositions of the present invention can have a drop point of at least about 30° C., at least about 40° C., or at least about 50° C. In some embodiments of the present invention, the solid fat compositions have a congeal point of between about 20° C. to about 40° C. For example, the solid fat compositions of the present invention can have a congeal point of at least about 20° C., at least about 25° C., or at least about 30° C. In some embodiments of the present invention, the solid fat compositions can have an iodine value of between about 50 to about 250. For example, the solid fat compositions of the present invention can have an iodine value of at least about 100, at least about 150, or at least about 200. In some embodiments of the present invention, the solid fat compositions can have a saponification value of between about 150 to about 275. For example, the solid fat compositions of the present invention can have a saponification value of between about 160 to about 260, between 170 to about 240, between about 180 to about 220, or between about 185 to about 215. In some embodiments of the present invention, the solid fat compositions of the present invention have less than about 0.5 ppm arsenic, less than about 0.04 ppm copper, less than about 0.1 ppm iron, less than about 0.2 ppm lead, and less than about 0.04 ppm mercury. In some embodiments of the present invention, the solid fat compositions of the present invention have a solid fat content profile of: between about 10% to about 50%, between about 12% to about 48%, or between about 15% to about 45% at 10.0° C.; between about 5% to about 35%, between about 7% to about 30%, or between about 10% to about 25% at 21.1° C.; between about 2% to about 25%, between about 4% to about 24%, or between about 6% to about 20% at 26.7° C.; between 0% to about 20%, between about 2% to about 18%, or between about 3% to about 16% at 33.3° C.; and between 0% to about 15%, between about 2% to about 14%, or between about 0.5% to about 12% at 37.8° C.

The oils used in the methods of the invention to produce a solid fat composition include an oil with at least one LC-PUFA. In some embodiments, the oil with at least one LC-PUFA is a microbial oil. Microbial sources and methods for growing microorganisms comprising nutrients and/or LC-PUFAs for recovery in microbial oils are known in the art, as described in detail above in the description of the minimally processed oils of the present invention. Such microbial sources and methods are suitable for producing microbial oils as a starting material for the solid fat compositions of the present invention. Indeed, minimally processed oils as described above are a preferred starting material for production of solid fat compositions. It should be appreciated, however, that a wide variety of other microbial oil starting materials, as described below, can be used as starting materials for solid fat compositions of the present invention. In one particularly preferred embodiment, the microbial oil is an oil produced according to the disclosures in PCT Patent Application Serial No. PCT/IB01/00841 entitled “Method for the Fractionation of Oil and Polar Lipid-Containing Native Raw Materials” filed Apr. 12, 2001, published as WO 01/76715 and PCT Patent Application Serial No. PCT/IB01/00963 entitled “Method for the Fractionation of Oil and Polar Lipid-Containing Native Raw Materials Using Water-Soluble Organic Solvent and Centrifugation” filed Apr. 12, 2001, published as WO 01/76385, the contents of which are incorporated herein by reference, in their entirety. Disclosures in these two PCT applications describe a microbial oil recovery process that can be generally referred to as the Friolex process.

Microbial oils suitable for use in the invention include at least one LC-PUFA. Preferred PUFAs of the present invention include C20, C22, or C24 omega-3 or omega-6 PUFAs. Preferably, the PUFA is an LC-PUFA, comprising a C20 or C22 omega-3, or a C20 or C22 omega-6 PUFA. An LC-PUFA of the present invention contains at least two double bonds and preferably, three double bonds, and even more preferably at least four double bonds. PUFAs having 4 or more unsaturated carbon-carbon bonds are also commonly referred to as highly unsaturated fatty acids, or HUFAs. In particular, the LC-PUFA can include docosahexaenoic acid (at least about 10, about 20, about 30, about 35, about 40, about 50, about 60, about 70 or about 80 weight percent of total fatty acids), docosapentaenoic acid n-3 (at least about 10, about 20, about 30, about 40, about 50, about 60, about 70 or about 80 weight percent of total fatty acids), docosapentaenoic acid n-6 (at least about 10, about 20, about 30, about 40, about 50, about 60, about 70 or about 80 weight percent of total fatty acids), arachidonic acid (at least about 10, about 20, about 30, about 40, about 50, about 60, about 70 or about 80 weight percent of total fatty acids) and/or eicosapentaenoic acid (at least about 10, about 20, about 30, about 40, about 50, about 60, about 70 or about 80 weight percent of total fatty acids). The PUFAs can be in any of the common forms found in natural lipids including but not limited to triacylglycerols, diacylglycerols, monoacylglycerols, phospholipids, free fatty acids, esterified fatty acids, or in natural or synthetic derivative forms of these fatty acids (e.g. calcium salts of fatty acids, ethyl esters, etc). In preferred embodiments, the microbial oils comprise at least about 70 wt. % of the PUFAs in the oil in the triglyceride form, at least about 80 wt. %, at least about 90 wt. %, and at least about 95 wt. %. The term LC-PUFA, as used in the present invention, can refer to either an oil comprising a single omega-3 LC-PUFA such as DHA, an oil comprising a single omega-6LC-PUFA such as ARA or DPA n-6, or an oil comprising a mixture of two or more LC-PUFAs such as DHA, DPA n-6, ARA, and EPA. In preferred embodiments, the product comprises an LC-PUFA in combination with at least one other nutrient.

In preferred embodiments of the invention, the oil comprising at least one LC-PUFA used in methods of the invention to produce a solid fat composition can include about 5 wt. % to about 70 wt. % LC-PUFA. For example, in some embodiments, the oil can include at least about 5 wt. %, at least about 10 wt. %, at least about 15 wt. %, at least about 20 wt. % of LC-PUFA, at least about 25 wt. %, at least about 30 wt. %, at least about 35 wt. % of LC-PUFA, at least about 40 wt. %, at least about 45 wt. %, and at least about 50 wt. % of LC-PUFA. Such embodiments can also have less than about 30 wt. %, less than about 35 wt. %, less than about 40 wt. %, less than about 45 wt. %, less that about 50 wt. %, less than about 55 wt. %, less than about 60 wt. %, less than about 65 wt. %, and less than about 70 wt. % LC-PUFA.

The oils used in methods of the invention to produce a solid fat composition, in addition to an oil comprising at least one LC-PUFA, may optionally include saturated fat. Saturated fats will typically have a higher melting point than the LC-PUFA or mixture of LC-PUFAs. Such a saturated fat can be added to the oil exogenously. Preferred exogenously added saturated fats to add include “hard fats” such as partially hydrogenated vegetable oils, fully hydrogenated oils, partially hydrogenated lards, and non-trans tropical oils. For example, palm oil and palm kernel oil and fractions thereof (palm and palm kernel olein as well as palm and palm kernel stearin) can be used. When the composition includes an exogenously added fat, the LC-PUFA oil may or may not be winterized. A preferred amount of exogenously added fat can be determined by one of skill in the art depending on the degree of solidity and/or viscosity of the starting material and the desired degree of solidity and/or viscosity and/or spread consistency desired in the composition. Exogenously added fats can be added in amounts of from about 20 wt. % to about 60 wt. %, from about 30 wt. % to about 50 wt. %, and from about 35 wt. % to about 45 wt. %.

In preferred embodiments, the saturated fat in the oil comprising at least one LC-PUFA is not added exogenously, but occurs naturally in the oil. For example, microbial oils comprising LC-PUFAs may be unprocessed oils extracted by any means known in the art. In such oils, the amount of saturated fats in the microbial oil can be from about 20 wt. % to about 60 wt. %, from about 30 wt. % to about 50 wt. %, and from about 35 wt. % to about 45 wt. %.

In preferred embodiments of the present invention, the oil comprising at least one LC-PUFA used is unwinterized (i.e., unfractionated) and will therefore contain saturated fats. Winterization refers to the process of removing sediment (typically, high melting solid saturated fats) that appears in many oils, including vegetable oils, at low temperature, most typically involving the removal of the quantity of crystallized material by filtration to avoid clouding of the liquid fractions at refrigerator temperatures. Such techniques include separating oils into two or more fractions with different melting points. The separated liquid and solid fractions exhibit significant differences in physical and chemical properties. Suitable techniques are known in the art and typically include the following three steps: (i) cooling of the liquid oil to supersaturation, resulting in the formation of nuclei for crystallization. (ii) progressive growth of the crystals by gradual cooling, and (iii) separation of the liquid and crystalline phases. These techniques can include, for example, conventional winterization, detergent fractionation and solvent winterization. Conventional winterization includes dry fractional crystallization wherein triglycerides with the highest melting temperature preferentially crystallize during cooling from the neat liquid or melted fat. The principle of dry fractionation process is based on the cooling of oil under controlled conditions without the addition of chemicals. The liquid and solid phases are separated by mechanical means. The principle of detergent fractionation is similar to dry fractionation based on the cooling of oil under controlled conditions without the addition of a solvent. Subsequently, the liquid and solid phases are separated by centrifugation after an aqueous detergent solution has been added. Solvent (typically acetone) winterization is used to promote triglyceride crystal formation, because triglycerides at low temperature generally form more stable crystals with solvent than without solvent. In solvent-aided fractionation, either polar or non-polar solvents may be used to reduce the viscosity of the system during filtration. The fractions obtained are then freed from the solvent by distillation. Thus, unwinterized microbial oils are those that have not been subjected to a winterization or fractionation process.

In further preferred embodiments, the oil comprising at least one LC-PUFA is not hydrogenated or partially hydrogenated. Hydrogenation is known in the art, and includes processes of chemically adding hydrogen gas to a liquid fat in the presence of a catalyst. This process converts at least some of the double bonds of unsaturated fatty acids in the fat molecules to single bonds thereby increasing the degree of saturation of the fat. The degree of hydrogenation, that is the total number of double bonds that are converted, determines the physical and chemical properties of the hydrogenated fat. An oil that has been partially hydrogenated often retains a significant degree of unsaturation in its fatty acids. Hydrogenation also results in the conversion of some cis double bonds to the trans configuration in which one or more double bonds has migrated to a new position in the fatty acid chain. Current studies indicate that trans-fatty acids may raise total cholesterol and heart disease risk to about the same extent as saturated fatty acids and are, therefore, undesirable in the diet. The present invention allows for the formation of a solid fat product without the need for hydrogenation or partial hydrogenation.

The oil comprising saturated fat used in the present invention can be in a solid, semi-solid, or liquid form. A variety of oils with saturated fat can be suitably used for producing the solid fat compositions of the present invention. In some embodiments, the oil comprising saturated fat includes, but is not limited to, microbial stearin, unfractionated palm oil, palm olein, palm stearin, palm mid fraction, unfractionated palm kernel oil, palm kernel olein, palm kernel stearin, unfractionated cotton seed oil, cotton seed olein, cotton seed stearin, coconut oil, unfractionated shea butter oil, shea butter stearin, interesterified palm oil blend, interesterified cotton seed oil blend, fish oil stearin (such as menhaden oil stearin), and combinations thereof. In preferred embodiments of the present invention, the oil comprising saturated fat is not hydrogenated or partially hydrogenated.

The present methods do not require the use of an emulsifier in producing a stable solid fat composition. However, an emulsifier may optionally be used in certain embodiments. Emulsifiers suitable for use with the present invention include a monoglyceride, a diglyceride, a mono/diglyceride combination, a lecithin, a lactylated mono-diglyceride, a polyglycerol ester, a sucrose fatty acid ester, sodium steroyl lactylate, calcium steroyl lactylate, and combinations thereof. In some embodiments, the emulsifier is a mono/diglyceride combination. In some embodiments, the emulsifier is present in the mixture in an amount of between about 0.01 weight percent and about 2.0 weight percent, in an amount of between about 0.025 weight percent and about 1.0 weight percent, and in an amount of between about 0.05 weight percent and about 0.2 weight percent. In a preferred embodiment of the present invention, the emulsifier is present in less than 0.01%, less than 0.009%, less than 0.005%, or less than 0.002% weight percent. In particularly preferred embodiments of the present invention, no exogenous emulsifier is added in producing the solid fat compositions.

It is suggested that an emulsifier may act to provide stability between various components in the mixture to maintain a homogeneous composition. Lack of stability may result in separation of oils or separation of the oil and a water phase. Emulsifiers may also provide functional attributes in addition to emulsification, which include aeration, starch and protein complexing, hydration, crystal modification, solubilization, and dispersion. The inventors, however, have surprisingly found that a stable, homogenous solid fat composition can be produced without the use of an emulsifier, as described herein.

The physical step of mixing an oil comprising saturated fat and an oil comprising at least one LC-PUFA may be conducted in any conventional manner of mixing known in the art. The compositions are mixed to achieve mixing, such as to achieve a homogeneous liquid solution. It is possible to heat the oil comprising saturated fat and/or the oil comprising at least one LC-PUFA (for example, to at least about 40° C.), so that the compositions are completely liquid and miscible in each other. However, the inventors have found that heating of the oils prior to mixing is unnecessary to form a homogeneous solid fat composition. Without being bound by theory, the inventors believe that the heat from at least the subsequent deodorization step will facilitate the homogenization of the oil mixture to form a homogenous solid fat product such that heating the oils prior to mixing is unnecessary. Therefore, in a preferred embodiment, the oil comprising saturated fat and/or the oil comprising at least one LC-PUFA are not heated prior to mixing. The ability to avoid heating the oils prior to mixing advantageously simplifies the process of producing solid fat compositions and contributes to the conservation of energy and resources.

The present methods also include solidifying the mixture of the oil comprising saturated fat and the oil comprising at least one LC-PUFA to form a solid fat composition. For example, in an embodiment in which the mixture is above room temperature, the mixture can be allowed to cool to room temperature. Alternatively, the mixture can be actively cooled to room temperature or, for example, below room temperature. For example, the composition can be cooled to between about 25° C. to about 30° C. to solidify. During the step of cooling, whether active or passive, the mixture can be mixed or agitated. In this manner, cooling can be controlled so that uniform cooling is achieved without creating a stratified composition. Preferably, such cooling conditions are adjusted in order to allow the crystal structure of the fat (i.e., the manner in which the molecules orient themselves in the solid stage) to reach desired levels, resulting in desired product plasticity, functionality, and stability. In general, β-prime crystals result in a smooth, creamy consistency. β crystals are typically larger, coarser and grainier than β-prime crystals, and are typically less desirable. Accordingly, in preferred embodiments, the cooling process is controlled so as to allow triglycerides in the mixture to reach stable, β-prime configurations to produce a product having a smooth consistency. Methods to cool that allow such preferred crystallization forms include cooling the mixture at a rate of between about 1° C./min and about 20° C./min, between about 5° C./min and about 15° C./min, and at about 10° C./min. Preferably, at least about 50 wt. % of the fats and/or oils in the solid fat composition, at least about 55 wt. %, at least about 60 wt. %, at least about 65 wt. %, at least about 70 wt. %, at least about 75 wt. %, at least about 80 wt. %, at least about 85 wt. %, at least about 90 wt. %, at least about 95 wt. %, or about 100 wt. % are in the β-prime crystal configuration.

In preferred embodiments, the solid fat composition of the present invention has a homogeneous texture and, therefore, has a uniform appearance and consistency. Another characteristic of these embodiments is that the composition is stable, and does not separate upon standing or otherwise lose its homogeneous texture, preferably for extended periods of time. Thus, the composition does not develop a non-uniform appearance or consistency upon standing. In preferred embodiments, the composition of the present invention can stand at least about one day, at least about one week, at least about two weeks, at least about three weeks, and at least about four weeks at room temperature without separating or otherwise losing its homogeneous texture.

The solid fat compositions of the present invention are a rich source of LC-PUFAs. In some embodiments, the solid fat composition comprises at least about 15 weight percent, at least about 20 weight percent, at least about 25 weight percent, or at least about 30 weight percent of at least one LC-PUFA, particularly docosahexaenoic acid. In preferred embodiments of the present invention, the solid fat compositions are free of trans-fatty acids.

The present invention also provides a solid fat composition comprising a mixture of a stearin composition comprising at least one LC-PUFA and a second oil comprising saturated fat, wherein the composition is solid at room temperature. In some embodiments of the present invention, a method for producing such a solid fat composition comprises mixing a stearin comprising at least one LC-PUFA with a second oil comprising saturated fat to form a mixture and solidifying the mixture to form a solid fat composition. Suitable stearin include, but is not limited to, microbial stearin, fish oil stearin, palm stearin, palm kernel stearin, cotton seed stearin, shea butter stearin, and combinations thereof. The second oils comprising saturated fat which are suitable for use in the present invention include, but are not limited to, unfractionated palm oil, palm olein, unfractionated palm kernel oil, palm kernel olein, palm mid fraction, coconut oil, unfractionated shea butter oil, unfractionated cotton seed oil, cotton seed olein, interesterified palm oil blend, interesterified cotton seed oil blend, and combinations thereof. Emulsifiers described herein may optionally be used in the formation of such solid fat compositions of the present invention.

The compositions of the present invention can also include a number of additional functional ingredients. For example, the compositions of the present invention can further include microencapsulants including, for example, proteins, simple and complex carbohydrates, solids and particulates. Preferred microencapsulants include: cell particulates; gum acacia; maltodextrin; hydrophobically modified starch; polysaccharides including alginate, carboxymethylcellulose and guar gum; hydrophobically-modified polysaccharides such as octyl-substituted starches; proteins including whey protein isolates, soy proteins, and sodium caseinate; and combinations thereof. In addition, compositions of the invention can include surfactants, including for example, anionic agents, cationic agents, nonionic agents, amphoteric agents, water-insoluble emulsifying agents, finely divided particles and naturally occurring materials. Anionic agents include carboxylic acids, sulfuric esters, alkane sulfonic acids, alkyl aromatic sulfonic acids, and miscellaneous anionic hydrophilic groups. Cationic agents include amine salts, ammonium compounds, other nitrogenous bases, and non-nitrogenous bases. Nonionic agents include an ether linkage to solubilizing group, ester linkage, amide linkage, miscellaneous linkage, and multiple linkages. Amphoteric agents include amino and carboxy, amino and sulfuric esters, amino and alkane sulfonic acids, amino and aromatic sulfonic acids, and miscellaneous combinations of basic and acidic groups. Water insoluble emulsifying agents include ionic hydrophilic groups and nonionic hydrophilic groups. Finely divided particles include any finely divided non-solubilized particle including clays and carbon. Naturally occurring materials include alginates, cellulose derivatives water-soluble gums, lipids and sterols, phospholipids, fatty acids, alcohols, proteins, amino acids, and detergents. Compositions of the present invention can also include hydrophilic colloids. Other optional ingredients include thickening agents such as polysaccharides. Thickeners are ingredients that are used to increase the viscosity of the composition. In such embodiments, the additional functional ingredient(s) are added during the step of mixing.

In one embodiment, the solid fat compositions are shortening. Shortenings typically have little to no added water or aqueous component and comprise high levels of fats. Alternatively, the solid fat compositions can be products such as a margarine, spread, mayonnaise, or salad dressing. Such products are prepared by blending fats and/or oils with other ingredients such as water and/or milk products, suitable edible proteins, salt, flavoring and coloring materials and Vitamins A and D. Margarine typically contains at least 80% fat. Mayonnaise and salad dressing are semi-solid fatty foods that typically contain not less than 65% and 30% vegetable oil, respectively, and dried whole eggs or egg yolks. Salt, sugar, spices, seasoning, vinegar, lemon juice, and other ingredients complete these products.

Accordingly, the compositions of the present invention can further include additional ingredients. Preferred additional ingredients include antioxidants, flavors, flavor enhancers, sweeteners, pigments, vitamins, minerals, pre-biotic compounds, pro-biotic compounds, therapeutic ingredients, medicinal ingredients, functional food ingredients, processing ingredients, and combinations thereof.

In a particularly preferred embodiment, the additional ingredient is an antioxidant. Antioxidants are known in the art, and may be added at any point in the production of the microbial oil by fermentation or lipid processing, or during the processes of the present invention. Antioxidants can help to preserve the resulting products from oxidative deterioration.

Suitable antioxidants may be chosen by the skilled artisan. Preferred antioxidants include ascorbyl palmitate, tocopherols, citric acid, ascorbic acid, tertiary butyl hydroquinone (TBHQ), butylated hydroxyanisole (BHA), butylated hydroxytoluene (BHT), propyl gallate (PG), rosemary extract, lecithin, folic acid, and mixtures and salts thereof. Antioxidants can be included in products in amounts that are conventional in the art.

The oxidative state and stability of a composition including a lipid may be measured in a number of ways known in the art, and descriptions of many of these techniques are available from the American Oil Chemist's Society, as well as from other sources. One method of quantifying the oxidative stability of a product is by measuring the Rancimat Value that measures the amount of conductive species (volatile decomposition products) that are evolved from a sample as it is subjected to thermal decomposition. In preferred embodiments, compositions of the present invention have Rancimat values of at least about 10 hours, at least about 15 hours, at least about 20 hours, and at least about 25 hours, at a temperature of 91.6° C.

In preferred embodiments, the products of the present invention (including the high quality PUFA-containing oil products and the solid fat compositions) are stored under appropriate conditions to minimize oxidative degradation. Many methods to effect such storage conditions are known in the art and are suitable for use with the present invention, such as, for example, replacement of ambient air with an inert gas atmosphere. A preferred method by which to reduce or minimize oxidative degradation is to store products under a nitrogen (N₂) or argon atmosphere or mixed nitrogen and carbon dioxide atmosphere. Preferably, packaged products are packaged under nitrogen. Methods for producing a nitrogen gas atmosphere into a container comprising a product are known in the art. In other preferred embodiments, oxidative and/or chemical stability of the products can also be increased by bubbling nitrogen into the mixture as it is cooling to provide extra protection against oxidation.

In another preferred embodiment, products of the present invention can comprise a pharmaceutically acceptable excipient and/or an added pharmaceutically active agent (i.e., a therapeutically or medicinally active ingredient or combinations thereof). This embodiment is particularly advantageous for pharmaceutically active agents that have low solubility in water. Such pharmaceutical products have the advantage of providing therapeutically active ingredients together with beneficial nutrients such as LC-PUFAs. Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Pharmaceutically active agents of the present invention include, without limitation, statins, anti-hypertensive agents, anti-diabetic agents, anti-dementia agents, anti-depressants, anti-obesity agents, appetite suppressants and agents to enhance memory and/or cognitive function. In another preferred embodiment, products of the present invention can comprise food ingredients such as functional food ingredients, food additives or other ingredients.

The products of the present invention can be used alone as a food product, nutritional product, or pharmaceutical product, or may be incorporated or added to a food, nutritional, or pharmaceutical product. In a first embodiment, the product of the invention is a food product that includes an oil product of the present invention and a food component. The products can be used directly as a food ingredient, such as an oil and/or shortening and/or spread and/or other fatty ingredient in beverages, sauces, dairy-based foods (such as milk, yogurt, cheese and ice-cream) and baked goods; or alternately used as a nutritional product, e.g., as a nutritional supplement (in capsule or tablet forms); feed or feed supplement for any animal whose meat or products are consumed by humans; feed or feed supplement for any companion animal, including without limitation dogs, cats, and horses; food supplement, including baby food and infant formula. The term “animal” means any organism belonging to the kingdom Animalia and includes, without limitation, any animal from which poultry, meat, seafood, beef, pork or lamb is derived. Seafood is derived from, without limitation, fish, shrimp and shellfish. The term “products” includes any product other than meat derived from such animals, including, without limitation, eggs, milk or other products. When fed to such animals, nutrients such as LC-PUFAs can be incorporated into the flesh, milk, eggs or other products of such animals to increase their content of these nutrients. In addition, when fed to such animals, nutrients such as LC-PUFAs can improve the overall health of the animal.

The compositions of the present invention can be added to a wide range of products such as baked goods, vitamin supplements, diet supplements, powdered drinks, etc. at various stages of production. Numerous finished or semi-finished powdered food products can be produced using the compositions of the present invention.

A partial list of food products comprising the products of the present invention includes: doughs; batters; baked food items including, for example, such items as cakes, cheesecakes, buns, tortillas, pies, cupcakes, cookies, bars, breads, rolls, biscuits, muffins, pastries, scones, and croutons; liquid food products, for example, beverages, energy drinks, infant formula, liquid meals, fruit juices, multivitamin syrups, meal replacers, medicinal foods, and syrups; semi-solid food products such as baby food, yogurt, cheese, cereal, pancake mixes; food bars including energy bars; processed meats; ice creams; frozen desserts; frozen yogurts; waffle mixes; salad dressings; and replacement egg mixes. Also included are: baked goods such as cookies, crackers, sweet goods, snack cakes, pies, granola/snack bars, and toaster pastries; salted snacks such as potato chips, corn chips, tortilla chips, extruded snacks, popcorn, pretzels, potato crisps, and nuts; specialty snacks such as dips, dried fruit snacks, meat snacks, pork rinds, health food bars and rice/corn cakes; and confectionary snacks such as candy, and cookie and cake filling.

Another product embodiment of the present invention is a medical food. A medical food includes a food which is in a formulation to be consumed or administered externally under the supervision of a physician and which is intended for the specific dietary management of a disease or condition for which distinctive nutritional requirements, based on recognized scientific principles, are established by medical evaluation.

The present invention, while disclosed in terms of specific methods, products, and organisms, is intended to include all such methods, products, and organisms obtainable and useful according to the teachings disclosed herein, including all such substitutions, modifications, and optimizations as would be available to those of ordinary skill in the art. When sources and amounts or ranges of the fatty acids and other ingredients are used herein, all combinations and subcombinations and specific embodiments therein are intended to be included. The following examples and test results are provided for the purposes of illustration and are not intended to limit the scope of the invention.

### EXAMPLES

### Example 1

### Preparation of High Quality Crude Oil

DHA oil-rich Schizochytrium microorganisms were grown in a fermentor to produce a fermentation broth. The fermentation broth was harvested and contacted with Alcalase®2.4, a protease that lysed the Schizochytrium cells. The resulting lysed cell mixture was an emulsion and was contacted with a 27% solution of isopropanol in water. This mixture was mixed by agitation and then subjected to centrifugation to produce a substantially non-emulsified product having two phases. The heavy phase contained components of the spent fermentation broth, and the light phase contained DHA-rich oil with some isopropanol and water. The light phase was dried to produce a high quality crude oil.

### Example 2

### Minimal Processing of Algal Oil

This example illustrates the production of minimally processed oils according to the present invention.

Minimally processed oils were produced in large scale. Two hundred kg of high quality crude oil produced as described in Example 1 by a Schizochytrium microorganism containing DHA was heated to 65° C. to 70° C. under nitrogen. About 0.2% (w/w of oil) of a 50% citric acid solution was then added into the oil and mixed for 30 to 45 minutes under nitrogen. Subsequently, 0.2 to 0.5% (w/w of oil) filter aid was added into the oil and filtered in order to remove any impurities present in oil. The oil was then deodorized at 210° C. with a feed rate of 180 kg per hour. Deodorized oil was then supplemented with tocopherols, ascorbyl palmitate and rosemary extract. Characteristics of oils at each process step are given in Table 1. The term “PV” means peroxide value; the term “FFA” means free fatty acid; and the term “p-AV” means p-anisidine value. Recovery from this process was greater than 98%.

TABLE 1

| | PV | FFA | | Phosphorus | DHA |
|---|---|---|---|---|---|
| Process Step | (meq/kg) | (%) | p-AV | (ppm) | (% w/w) |
| Crude | 0.15 | 0.22 | 3.7 | 3.32 | 34.0 |
| Citric acid-treated | 0.26 | 0.21 | 3.6 | below detection | Not |
| | | | | | analyzed |
| Deodorized | 0.28 | 0.13 | 4.9 | below detection | Not |
| without | | | | | analyzed |
| antioxidants | | | | | |
| Deodorized with | 0.0 | 0.15 | 4.0 | below detection | 33.2 |
| antioxidants | | | | | |

### Example 3a

### Physical Refining

This example illustrates the production of minimally processed oils according to the present invention.

Approximately 600 kg of high quality crude oil (produced as described in Example 1; FFA<0.3%, Phosphorus<10 ppm, PV<2 meq/kg) was heated to 50-55° C. under nitrogen and/or vacuum. About 0.2% (w/w) of 50% citric acid was added and the oil was held at 50-55° C. under nitrogen and/or vacuum for 15 minutes. Trisyl 600 (0.1%-3% w/w, usually 0.25%) was added and the temperature was held between 50-55° C. under nitrogen and/or vacuum for 15 minutes. Tonsil Supreme FF bleaching clay (0.1%-4% w/w, usually less than 0.5%) was added and the oil was heated to 90-95° C. and held under vacuum (>24″ Hg) for 30 minutes. Celite (0.1-0.5% w/w, usually 0.2%) was then added and the oil was filtered through a Sparkler filter. The oil was then deodorized at 210-225° C. and a flowrate of 180-225 kg/hr. After dcodorization, antioxidants were added. This process yielded an oil that is a semi-solid at room temperature.

Oil yields from this process ranged from −92-97%. Quality data for these runs with antioxidants are shown in Table 2

TABLE 2

| | Initial | Final | | | Initial | Final |
|---|---|---|---|---|---|---|
| | FFA | FFA | Initial PV | Final PV | Phos. | Phos. |
| Trial No. | (%) | (%) | (meq/kg) | (meq/kg) | (ppm) | (ppm) |
| Trial #1 | <0.1 | 0.11 | 1.15 | 0 | 9.2 | 1.9 |
| Trial #2 | <0.1 | 0.09 | 0.15 | 0 | 5.6 | 0 |
| Trial #3 | 0.28 | 0.19 | 0.25 | <0.1 | 2.6 | 3.4 |
| Trial #4 | 0.23 | 0.21 | 0.26 | 0 | 3.3 | 0 |

FFAs of deodorized oils were measured before and after antioxidants addition. A significant increase in FFAs (about 2×) was observed after adding antioxidants.

### Example 3b

### Physical Refining (Clear Oil)

This example illustrates the production of minimally processed liquid oils and related solid fat products according to the present invention.

Approximately 1200 kg of high quality crude oil (produced as described in Example 1; FFA<0.3%, Phosphorus<12 ppm, PV<2 meq/kg) was heated to 50-55° C. under nitrogen and/or vacuum. About 0.2% (w/w) of 50 wt % citric acid was added and the oil was held at 50-55° C. under nitrogen and/or vacuum for 15 minutes. The oil was then chilled from −55° C. to −35° C. under nitrogen and/or vacuum using various hold times (0-12 hrs.) and agitator speeds (4-16 rpm). At this time, celite (0.1-0.5% w/w, usually 0.2%) was added and the oil was filtered through a Sparkler filter. The chill filtration step was repeated with the oil being heated under nitrogen and/or vacuum and chilled from −50° C. to −30° C. using various hold times (0-12 hrs.) and agitator speeds (4-16 rpm). Celite (0.1-0.5% w/w, usually 0.2%) was added again and the oil was filtered through a Sparkler filter. Next, Trisyl 600 (0.1%-3% w/w, usually 0.25%) was added and the temperature was held between 50-55° C. under nitrogen and/or vacuum for 15 minutes. Tonsil Supreme FF bleaching clay (0.1%-4% w/w, usually 0.5% or less) was added and the oil was heated to 90-95° C. and held under vacuum (>24″ Hg) for 30 minutes. Celite (0.1-0.5% w/w, usually 0.2%) was added and the oil was filtered through a Sparkler filter. The oil was then chilled again under nitrogen and/or vacuum from −40° C. to −20° C. using various hold times (0-12 hrs.) and agitator speeds (4-16 rpm). Celite (0.1-0.5% w/w, usually 0.2%) was added and the oil was filtered through a Sparkler filter. The oil was then deodorized at 210-225° C. and a flowrate of 180-225 kg/hr. After deodorization, antioxidants were added. This yields an oil that is clear at room temperature. Oil yields from this process range from −55-60%. Quality data for these runs with antioxidants are shown in Table 3.

TABLE 3

| | Initial | Final | | | Initial | Final |
|---|---|---|---|---|---|---|
| | FFA | FFA | Initial PV | Final PV | Phos. | Phos. |
| Trial No. | (%) | (%) | (meq/kg) | (meq/kg) | (ppm) | (ppm) |
| Trial #1 | 0.21 | 0.1 | 0.32 | 0.5 | <5 | 2.6 |
| Trial #2 | 0.19 | 0.17 | <0.1 | 0.07 | 11 | 3.1 |
| Trial #3 | 0.12 | 0.17 | 0.53 | 0.07 | 3 | 6.5 |
| Trial #4 | 0.18 | 0.08 | 0.26 | 0 | 3.3 | 0.5 |

The material retained by the filter can be treated, for example by heating and filtering, to separate the solid material from the bleaching clay. Heating the material retained by the filter will melt the solids. The melted solids can then be separated from the clay, by filtering, for example, and then resolidified by cooling. The recovered solid will contain about 20-30% PUFA, most of which is DHA. The clear oil and the solid can be used as a food or food additive, for example.

### Example 3c

### Physical Refining/Silica Refining

This example illustrates the production of minimally processed oils according to the present invention.

Approximately 100 g of high quality crude oil (produced as described in Example 1; FFA

<0.8%, Phosphorus<10 ppm, PV<2 meq/kg) was heated to 50-55° C. under nitrogen. About 0.2% (w/w) of 50 wt % citric acid was added and the oil was held at 50-55° C. under nitrogen and/or vacuum for 15 minutes. Subsequently, 0.5%-1.25% w/w of silica (Brightsorb F100) was added and the oil was heated to 85° C. under vacuum. After 30 minutes holding time, Tonsil Supreme FF bleaching clay (0.5% w/w) was added, the oil was heated to 90-95° C. and held under vacuum (>24″ Hg) for 30 minutes. Celite (0.1-0.5% w/w, usually 0.2%) was then added and the oil was vacuum filtered using a Buchner funnel after cooling to 60-65° C. Yields for these tests were between 95-96%. Quality results for these tests are shown in Table 4. The final product was a semi-solid oil. This product could also be deodorized and/or bleached and would remain a semi-solid oil.

TABLE 4

| | | Initial | Final | Initial | Final | | |
|---|---|---|---|---|---|---|---|
| | | FFA | FFA | PV | PV | Initial | Final |
| Trial No. | % Silica | (%) | (%) | (meq/kg) | (meq/kg) | AV | AV |
| Trial #1 | 0.5% | 0.64 | 0.43 | 1.51 | 1.40 | 6.1 | n/a |
| Trial #2 | 0.8% | 0.64 | 0.34 | 1.51 | 1.33 | 6.1 | n/a |
| Trial #3 | 1.2% | 0.64 | 0.17 | 1.51 | 1.33 | 6.1 | 6.3 |

### Example 3d

### Modified Caustic Refining

This example illustrates the production of minimally processed oils according to the present invention.

Approximately 600 kg of high quality crude oil (produced as described in Example 1; with FFA up to 0.8% Phosphorus<12 ppm. PV<2 meq/kg) was heated to 50-55° C. under nitrogen and/or vacuum. About 0.2% (w/w) of 50 wt % citric acid was added and the oil was held at 50-55° C. under nitrogen and/or vacuum for 15 minutes. At this time, 0.1%-0.5% w/w of 50% caustic was added to the oil and held at 60-65° C. for 15-30 minutes (this is −2-10 times less caustic than the standard amount used). The oil was then centrifuged to remove the soaps from the oil. Trisyl 600 (0.1%-3% w/w, usually 0.25%) was added and the temperature was held between 50-55° C. under nitrogen and/or vacuum for 15 minutes. Tonsil Supreme FF bleaching clay (0.1%-4% w/w, usually 0.5% or less) was added and the oil was heated to 90-95° C. and held under vacuum (>24″ Hg) for 30 minutes. Celite (0.1-0.5% w/w, usually 0.2%) was added and the oil was filtered through a Sparkler filter. The oil was then deodorized at 210-225° C. and a flowrate of 180-225 kg/hr. After deodorization, antioxidants were added. This process yielded a semi-solid liquid.

Oil yields from this process range from −81-91%. Quality data for these runs with antioxidants are shown in Table 5.

TABLE 5

| | Initial | Final | Initial | Final | Initial | Final |
|---|---|---|---|---|---|---|
| Trial | FFA | FFA | PV | PV | Phos. | Phos. |
| No. | (%) | (%) | (meq/kg) | (meq/kg) | (ppm) | (ppm) |
| Trial #1 | 0.26 | <0.1 | 1.37 | 0 | 11.6 | 4.0 |
| Trial #2 | 0.54 | <0.1 | 1.84 | 0 | 9.8 | 4.5 |
| Trial #3 | 0.75 | 0.1 | 0.17 | <0.1 | 8.0 | 5.0 |
| Trial #4 | 0.40 | 0.13 | 0 | <0.1 | 7.0 | 0.6 |
| Trial #5 | 0.23 | 0.08 | 0.31 | 0 | 3.3 | 0.9 |

### Example 3e

### Modified Caustic Refining/No Centrifugation

This example illustrates the production of minimally processed oils according to the present invention.

Approximately 100 g of high quality crude oil (produced as described in Example 1; FFA

<0.3%, Phosphorus<10 ppm, PV<2 meq/kg) was heated to 50-55° C. under nitrogen and/or vacuum. About 0.2% (w/w) of 50 wt % citric acid was added and the oil was held at 50-55° C. under nitrogen and/or vacuum for 15 minutes. At this time, 0.4% w/w of a 50% caustic solution was added to the oil and held at 60-65° C. for 15-30 minutes (this is −2-10 times less caustic solution than the standard amount used). Next, Trisyl 600 (1.5% w/w) was added and the temperature was held between 50-55° C. under nitrogen and/or vacuum for 15 minutes. Celite (0.2% w/w) was added to the oil and it was vacuum filtered using a Buchner funnel. Tonsil Supreme FF bleaching clay (1.0% w/w) was added to the filtered oil and it was heated to 90-95° C. and held under vacuum (>24″ Hg) for 30 minutes. Celite (0.2% w/w) was added and the oil was vacuum filtered using a Buchner funnel. Quality results for this test are shown in Table 6. The final product was a semi-solid oil. This product could also be deodorized and/or bleached and would remain a semi-solid oil.

TABLE 6

| | Initial | Final | Initial | Final | | |
|---|---|---|---|---|---|---|
| Trial | FFA | FFA | PV | PV | Initial | Final |
| No. | (%) | (%) | (meq/kg) | (meq/kg) | AV | AV |
| Trial #1 | 0.64 | 0.14 | 1.51 | 1.21 | 6.1 | 5.6 |

### Example 4

### Dry Fractionation of Crude Algal Oil

This example illustrates the dry fractionation of crude algal oil containing DHA produced by a Schizochytrium microorganism into olein and stearin fractions according to the present invention.

Three hundred and fifty kg of the crude oil was subjected to the dry fractionation process according to the present invention in order to produce liquid olein and solid stearin fractions. Melting of all crystalline phases within the crude algal oil was ensured by heating the same to 60-70° C. in a vessel with stirring. The material was then cooled rapidly to 20-30° C. during the pre-cooling phase, with the speed of the stirrer increased to 40 revolutions per minute. In order to obtain the highest possible heat transfer coefficient in this phase, a liquid coolant was employed, which was water in this example. The temperature of the coolant was not permitted to fall significantly below the nucleation temperature.

The subsequent nucleation phase was conducted within the stirring vessel and was initiated by a reduction of the stirrer speed to 20 revolutions per minute. Further cooling of the oil was done by regulating the temperature difference between the coolant and the oil, from an initial oil temperature of 20-30° C., down to the crystallization temperature of about 12-14° C. Once the crystallization temperature had been reached, the stirrer speed was reduced to 15 revolutions per minute. Termination of the crystallization was accomplished by transferring the suspension into a filtration unit immediately after the desired cloud point was reached for the remaining oil, with the olein fraction present between the crystals. To monitor the cloud point of the olein fraction, test filtrations of suspension samples were performed during the crystallization phase.

After the crystal suspension has been transferred to the filtration unit, the liquid phase was pressed out through a filter cloth. The filter chamber was charged with a slowly increasing compression pressure that was generated by a mechanical reduction of the volume of the filter chamber, and was slowly increased. The final filtration pressure reached 10 bar. After filtration, the separated fractions were weighed. The olein yield is the weight of the filtrate. The stearin yield is the weight of the crystal mass remaining on the filter. The yields of the measured olein and stearin fractions are given in Table 7. The compositions of the feed materials, olein and stearin fractions are given in Table 8.

TABLE 7

| Parameter | Results |
|---|---|
| Cooling curve (h) | 13 |
| Final Temperature of the Slurry (C) | 14.2 |
| Solid Fat Content of the Slurry (%) | 7.3 |
| Solid Fat Content of the Stearin (%) | 39.6 |
| Olein Yield (%) | 83.4 |
| Stearin Yield (%) | 14.4 |

TABLE 8

| Parameter | Feed | Olein | Stearin |
|---|---|---|---|
| Moisture content (ppm) | 564-660 | — | — |
| Cloud point (° C.) | 11.5-17.4 | −4.8 to −5.5 | — |
| Iodine value | 235.8-265 | 2604-278.7 | 184.2-210.8 |
| Fatty acid composition | | | |
| (% w/w): | | | |
| 12:0 | 0.2-0.4 | 0.3-0.4 | 0.3-0.6 |
| 14:0 | 10.0-12.6 | 8.6-8.8 | 14.9-16.1 |
| 14:1 | 0.4-0.5 | 0.0-0.4 | 0.5-0.6 |
| 16:0 | 25.3-27.1 | 22.5-23.1 | 36.1-39.1 |
| 16:1 | 0.7-0.8 | 0.0 | 0.0 |
| 18:1n-9 | 0.3-1.9 | 0.3-0.5 | 0.0-0.4 |
| 22:1 | 0.9-1.0 | 1.0-1.1 | 0.7-0.8 |
| 20:5n-3 | 1.4-1.6 | 1.7-1.8 | 1.0-1.5 |
| 22:5n-6 | 14.6-17.1 | 18.0-18.3 | 11.9-12.9 |
| 22:6n-3 | 39.8-43.4 | 45.8-46.0 | 29.1-31.8 |
| Solid fat content (%): | | | |
| 0° C. | 8.7 | 0.0 | 36.3-44.1 |
| 10° C. | 7.5 | — | 34.8-41.2 |
| 15° C. | 6.8 | — | 33.2-38.5 |
| 20° C. | 6.1 | — | 30.5-35.9 |
| 25° C. | 5.4 | — | 28.9-34.0 |
| 30° C. | 3.1 | — | 26.3-31.1 |
| 35° C. | 2.4 | — | 21.0-25.4 |
| 40° C. | 0.8 | — | 12.9-17.2 |
| 45° C. | 0.0 | — | 4.5-5.2 |
| 50° C. | 0.0 | — | 1.5-2.0 |
| 55° C. | 0.0 | — | 0.0 |

The olein (liquid) and stearin (solid or semi-solid) fractions could be further processed to produced deodorized oil by any of the minimal processing methods described herein and illustrated in the above examples, or by any method known in the art.

### Example 5

The following Example shows a process for forming a solid fat product from a crude semi-solid oil and DHA-stearin (1:1 mass ratio).

Approximately 1 kg of crude DHA-stearin, produced by Schizochytrium microorganism containing DHA as a by-product from the winterization process, was vacuum-filtered to remove the filter aid introduced by the winterization process. Approximately 400 g of filtered DHA-stearin was then combined with 400 g of semi-solid crude oil produced by Schizochytrium microorganism containing DHA. This oil mixture was then heated to 50-55° C. under nitrogen. About 0.2% (w/w) of 50 wt % citric acid was added and the oil mixture was held at 50-55° C. under nitrogen for 15 minutes. After 15 minutes, the oil mixture was heated to 60-65° C. At this time, 0.45% (w/w of oil) of caustic solution (50% caustic solution and soft water, 1:3 w/w ratio) was added to the oil mixture and held at 60-65° C. for 15 minutes. After 15 minutes at 60-65° C., the oil mixture was heated to 80° C. and then centrifuged to remove soaps from the oil mixture. Next, Trisyl 600 (0.25% w/w) was added and the temperature was held between 50-55° C. under nitrogen and/or vacuum for 15 minutes. Subsequently, Tonsil Supreme FF bleaching clay (0.5% w/w) was added and the oil was heated to 90-95° C. and held under vacuum (>24″ Hg) for 30 minutes. Celpure (0.1% w/w) was then added and the oil was filtered under vacuum. The oil was then deodorized at 210° C. for 30 minutes. After deodorization, antioxidants were added. This yields a homogenous product that is solid at room temperature. After cooling to 30-40° C., the resulting crystallized fat was transferred to containers and stored. Quality characteristics of the final product are as follows:

TABLE 9
Physical and Chemical Properties of the Final Product from Example 5

| Parameter | Results |
|---|---|
| Chemical Analyses: | |
| DHA (mg/g) | 331.2 |
| PV (meq/kg) | 0.4 |
| p-AV | 1.4 |
| Trans Fatty Acids (%) | Not detected |
| Moisture & Volatiles (%) | 0.01 |
| FFA (%) | 0.05 |
| Unsaponifiable Matter (%) | 1.0 |
| Rancimat Value (h) | 15.5 |
| Elemental Analyses (mg/kg): | |
| As | <0.5 |
| Cu | <0.04 |
| Fe | 0.1 |
| Pb | <0.2 |
| Hg | <0.04 |
| Solid Fat Content (%): | |
| 10° C. | 14.6 |
| 21.1° C. | 11.0 |
| 26.7° C. | 9.2 |
| 33.3° C. | 6.0 |
| 37.8° C. | 2.3 |
| Fatty Acid Profile (% of total fatty acids): | |
| 12:0 | 0.3 |
| 14:0 | 12.1 |
| 16:0 | 29.5 |
| 16:1 | 0.4 |
| 18:0 | 0.8 |
| 18:1n-9 | 1.4 |
| 18:1n-7 | 0.2 |
| 18:2n-6 | 0.3 |
| 20:3n-6 | 0.4 |
| 20:4n-6 | 1.7 |
| 20:5n-3 | 1.0 |
| 22:5n-6 | 13.8 |
| 22:6n-3 | 36.2 |

### Example 6

Sensory evaluation of the final product produced in Example 5, was conducted by 9 trained panelists using descriptive sensory analysis method with 0-15 scale, 0 being none detected and 15 being very high intensity. The product had low overall aroma intensity with low intensity green/beany-like and herbal notes. The aromatics of this product had low medium overall intensity with predominantly herbal and low intensity green/beany-like notes. The herbal aftertaste was noted as well. No fishy or painty notes detected in the aroma and aromatics. Overall, both the aroma and aromatics and the intensities are within an acceptable range. Results are given in Table 10 below.

TABLE 10
Sensory Scores of the Final Product from Example 5.

| Attributes | Sensory Score |
|---|---|
| Aroma: | |
| Total Impact | 3 |
| Green/Beany | 1.5 |
| Nutty/Roasted/Vitamin | 0 |
| Fishy | 0 |
| Painty | 0 |
| Herbal | 1.5 |
| Other | 0 |
| Aromatics: | |
| Total Impact | 4.5 |
| Green/Beany | 1.5 |
| Nutty/Roasted/Vitamin | 0 |
| Fishy | 0 |
| Painty | 0 |
| Herbal | 3 |
| Other | 0 |
| Aftertaste | Herbal |

### Example 7

The following Example shows a process for forming a solid fat product from a crude semi-solid oil and crude palm kernel stearin (1:1 mass ratio).

Approximately 125 g of semi-solid crude oil containing DHA, produced by Schizochytrium microorganism, was combined with 125 g of crude palm kernel stearin (PKS). The oil mixture was then heated to 70° C. under nitrogen. About 0.1% (w/w) of 50 wt % citric acid was added and the oil was held at 70° C. under nitrogen for 10 minutes. After 10 minutes, 0.6% (w/w of oil) of caustic solution (50% caustic solution and soft water, 1:3 w/w ratio) was added to the oil and held at 70° C. for 5 minutes. After the 5 minute hold at 70° C., the oil was centrifuged to remove soaps from the oil. Next, Trisyl 600 (0.1% w/w) was added and the temperature was held between 50-55° C. under nitrogen and/or vacuum for 10 minutes. Subsequently, Tonsil Supreme FF bleaching clay (0.1% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes with 3% sparge steam. After deodorization, antioxidants were added. This yields a homogenous product that is solid at room temperature. After cooling to 30-40° C., the resulting crystallized fat was then transferred to containers and stored. Quality characteristics and physical properties of the final product are given in Table 11.

TABLE 11
Physical and Chemical Properties of the Final Product from Example 7

| Parameter | Results |
|---|---|
| DHA (mg/g) | 173.1 |
| PV (meq/kg) | 1.4 |
| p-AV | 2.8 |
| FFA (%) | 0.04 |
| Acid Value (mg KOH/g) | 0.06 |
| Saponification Value | 212.7 |
| Iodine Value | 116.7 |
| Wiley Melting Point (° C.) | 35.2 |
| Solid Fat Content (%): | |
| 10° C. | 47.4 |
| 21.1° C. | 29.4 |
| 26.7° C. | 10.9 |
| 33.3° C. | 0.1 |
| 37.8° C. | 0.0 |
| Fatty Acid Profile (% of total fatty acids): | |
| 10:0 | 1.2 |
| 12:0 | 26.8 |
| 14:0 | 17.2 |
| 16:0 | 19.0 |
| 16:1 | 0.2 |
| 18:0 | 1.6 |
| 18:1n-9 | 4.9 |
| 18:2n-6 | 0.9 |
| 20:4n-6 | 1.1 |
| 20:5n-3 | 0.7 |
| 22:5n-6 | 7.1 |
| 22:6n-3 | 18.0 |

### Example 8

The following Example shows a process for forming a solid fat product from a crude semi-solid oil and crude palm kernel stearin (3:1 mass ratio).

Approximately 500 g of semi-solid crude oil containing DHA, produced by Schizochytrium microorganism, was combined with 166.6 g of crude palm kernel stearin (PKS). The oil mixture was then heated to 70° C. under nitrogen. About 0.1% (w/w) of 50 wt % citric acid was added and the oil was held at 70° C. under nitrogen for 10 minutes. After 10 minutes, 0.6% (w/w of oil) of caustic solution (50% caustic solution and soft water, 1:3 w/w ratio) was added to the oil and held at 70° C. for 5 minutes. After the 5 minute hold at 70° C., the oil was centrifuged to remove soaps from the oil. Next, Trisyl 600 (0.1% w/w) was added and the temperature was held between 50-55° C. under nitrogen and/or vacuum for 10 minutes. Subsequently, Tonsil Supreme FF bleaching clay (0.1% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes with 3% sparge steam. After deodorization, antioxidants were added. This yields a homogenous product that is solid at room temperature. After cooling to 30-40° C., the resulting crystallized fat was transferred to containers and stored. Quality characteristics and physical properties of the final product are given in Table 12.

TABLE 12
Physical and Chemical Properties of the Final Product from Example 8

| Parameter | Results |
|---|---|
| Chemical Analyses: | |
| DHA (mg/g) | 273.5 |
| PV (meq/kg) | 0.0 |
| p-AV | 3.3 |
| FFA (%) | 0.08 |
| Rancimat Value (h) | 21.0 |
| Trans Fatty Acids (%) | Not detected |
| Acid Value (mg KOH/g) | 0.19 |
| Saponification Value | 199.6 |
| Iodine Value | 191.4 |
| Wiley Melting Point (° C.) | 29.2 |
| Dropping Point (° C.) | 31.1 |
| Congeal Point (° C.) | 21.4 |
| Elemental Analyses (mg/kg): | |
| As | <0.02 |
| Cu | <0.2 |
| Fe | 0.9 |
| Pb | <0.02 |
| Hg | <0.01 |
| Solid Fat Content (%): | |
| 10° C. | 24.4 |
| 21.1° C. | 10.4 |
| 26.7° C. | 4.1 |
| 33.3° C. | 0.0 |
| 37.8° C. | 0.0 |
| Fatty Acid Profile (% of total fatty acids): | |
| 10:0 | 0.7 |
| 12:0 | 13.9 |
| 14:0 | 13.8 |
| 15:1 | 0.3 |
| 16:0 | 22.0 |
| 16:1 | 0.4 |
| 18:0 | 1.1 |
| 18:1n-9 | 3.5 |
| 18:1n-7 | 0.2 |
| 18:2n-6 | 0.7 |
| 20:3n-6 | 0.3 |
| 20:4n-6 | 1.3 |
| 20:5n-3 | 0.8 |
| 22:5n-6 | 11.0 |
| 22:6n-3 | 28.7 |

### Example 9

The following Example shows a process for forming a solid fat product from a crude semi-solid oil and crude palm kernel stearin (6:1 mass ratio). Approximately 150 g of semi-solid crude oil containing DHA, produced by Schizochytrium microorganism, was combined with 25 g of crude palm kernel stearin (PKS). The oil mixture was then heated to 70° C. under nitrogen. About 0.1% (w/w) of 50 wt % citric acid was added and the oil was held at 70° C. under nitrogen for 10 minutes. After 10 minutes, 0.6% (w/w of oil) of caustic solution (50% caustic solution and soft water, 1:3 w/w ratio) was added to the oil and held at 70° C. for 5 minutes. After the 5 minute hold at 70° C., the oil was centrifuged to remove soaps from the oil. Next, Trisyl 600 (0.1% w/w) was added and the temperature was held between 50-55° C. under nitrogen and/or vacuum for 10 minutes. Subsequently, Tonsil Supreme FF bleaching clay (0.1% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes with 3% sparge steam. After deodorization, antioxidants were added. This yields a homogenous product that is solid at room temperature. After cooling to 30-40° C., the resulting crystallized fat was then transferred to containers and stored. Quality characteristics and physical properties of the final product are given in Table 13.

TABLE 13
Physical and Chemical Properties of the Final Product from Example 9

| Parameter | Results |
|---|---|
| Chemical Analyses: | |
| DHA (mg/g) | 297.1 |
| PV (meq/kg) | 0.0 |
| p-AV | 2.4 |
| FFA (%) | 0.06 |
| Solid Fat Content (%): | |
| 10° C. | 18.3 |
| 21.1° C. | 10.0 |
| 26.7° C. | 6.6 |
| 33.3° C. | 3.2 |
| 37.8° C. | 0.7 |
| Fatty Acid Profile (% of total fatty acids): | |
| 10:0 | 0.4 |
| 12:0 | 8.1 |
| 14:0 | 13.4 |
| 15:1 | 0.3 |
| 16:0 | 25.1 |
| 16:1 | 0.3 |
| 18:0 | 1.0 |
| 18:1n-9 | 3.1 |
| 18:2n-6 | 0.6 |
| 20:3n-6 | 0.3 |
| 20:4n-6 | 1.7 |
| 20:5n-3 | 0.8 |
| 22:5n-6 | 12.3 |
| 22:6n-3 | 31.2 |

### Example 10

The following Example shows a process for forming a solid fat product from a crude semi-solid oil and crude palm stearin (1:1 mass ratio).

Approximately 250 g of semi-solid crude oil containing DHA, produced by Schizochytrium microorganism, was combined with 250 g of crude palm stearin (PS). The oil mixture was then heated to 70° C. under nitrogen. About 0.1% (w/w) of 50 wt % citric acid was added and the oil was held at 70° C. under nitrogen for 10 minutes. After 10 minutes, 0.6% (w/w of oil) of caustic solution (50% caustic solution and soft water, 1:3 w/w ratio) was added to the oil and held at 70° C. for 5 minutes. After the 5 minute hold at 70° C., the oil was centrifuged to remove soaps from the oil. Next, Trisyl 600 (0.1% w/w) was added and the temperature was held between 50-55° C. under nitrogen and/or vacuum for 10 minutes. Subsequently, Tonsil Supreme FF bleaching clay (0.5% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes with 3% sparge steam. After deodorization, antioxidants were added. This yields a homogenous product that is solid at room temperature. After cooling to 30-40° C., the resulting crystallized fat was transferred to containers and stored. Quality characteristics and physical properties of the final product are given in Table 14.

TABLE 14
Physical and Chemical Properties of the Final Product
from Example 10

| Parameter | Results |
|---|---|
| DHA (mg/g) | 186.9 |
| PV (meq/kg) | 2.2 |
| p-AV | 1.5 |
| FFA (%) | 0.04 |
| Acid Value (mg KOH/g) | 0.06 |
| Saponification Value | 191.6 |
| Iodine Value | 141.3 |
| Wiley Melting Point (° C.) | 53.0 |
| Solid Fat Content (%): | |
| 10° C. | 43.5 |
| 21.1° C. | 29.4 |
| 26.7° C. | 22.7 |
| 33.3° C. | 16.5 |
| 37.8° C. | 13.4 |
| Fatty Acid Profile (% of total fatty acids): | |
| 12:0 | 0.3 |
| 14:0 | 6.3 |
| 16:0 | 40.6 |
| 16:1 | 0.2 |
| 18:0 | 3.2 |
| 18:1n-9 | 16.7 |
| 18:1n-7 | 0.3 |
| 18:2n-6 | 3.2 |
| 20:0 | 0.3 |
| 20:4n-6 | 1.1 |
| 20:5n-3 | 0.8 |
| 22:5n-6 | 7.4 |
| 22:6n-3 | 19.0 |

### Example 11

The following Example shows a process for forming a solid fat product from a crude semi-solid oil and crude palm stearin (6:1 mass ratio).

Approximately 900 g of semi-solid crude oil containing DHA, produced by Schizochytrium microorganism, was combined with 150 g of crude palm stearin (PS). The oil mixture was then heated to 70° C. under nitrogen. About 0.1% (w/w) of 50 wt % citric acid was added and the oil was held at 70° C. under nitrogen for 10 minutes. After 10 minutes, 0.6% (w/w of oil) of caustic solution (50% caustic solution and soft water, 1:3 w/w ratio) was added to the oil and held at 70° C. for 5 minutes. After the 5 minute hold at 70° C., the oil was centrifuged to remove soaps from the oil. Next, Trisyl 600 (0.1% w/w) was added and the temperature was held between 50-55° C. under nitrogen and/or vacuum for 10 minutes. Subsequently, Tonsil Supreme FF bleaching clay (0.5% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes with 3% sparge steam. After deodorization, antioxidants were added. This yields a homogenous product that is solid at room temperature. After cooling to 30-40° C., the resulting crystallized fat was transferred to containers and stored. Quality characteristics and physical properties of the final product are given in Table 15.

TABLE 15
Physical and Chemical Properties of the Final Product
from Example 11

| Parameter | Results |
|---|---|
| Chemical Analyses: | |
| DHA (mg/g) | 299.9 |
| PV (meq/kg) | 0.0 |
| p-AV | 0.4 |
| FFA (%) | 0.07 |
| Rancimat Value (h) | 17.3 |
| Trans Fatty Acids (%) | Not detected |
| Acid Value (mg KOH/g) | 0.2 |
| Saponification Value | 186.7 |
| Iodine Value | 214.7 |
| Wiley Melting Point (° C.) | 35.4 |
| Dropping Point (° C.) | 41.6 |
| Congeal Point (° C.) | 27.3 |
| Elemental Analyses (mg/kg): | |
| As | <0.02 |
| Cu | <0.2 |
| Fe | <0.5 |
| Pb | <0.02 |
| Hg | <0.01 |
| Solid Fat Content (%): | |
| 10° C. | 16.3 |
| 21.1° C. | 11.7 |
| 26.7° C. | 8.8 |
| 33.3° C. | 6.4 |
| 37.8° C. | 4.1 |
| Fatty Acid Profile (% of total fatty acids): | |
| 12:0 | 0.4 |
| 14:0 | 10.5 |
| 15:1 | 0.4 |
| 16:0 | 31.4 |
| 16:1 | 0.3 |
| 18:0 | 1.5 |
| 18:1n-9 | 6.4 |
| 18:1n-7 | 0.2 |
| 18:2n-6 | 1.4 |
| 20:3n-6 | 0.3 |
| 20:4n-6 | 1.6 |
| 20:5n-3 | 1.0 |
| 22:5n-6 | 11.8 |
| 22:6n-3 | 31.5 |

### Example 12

The following Example shows a process for forming a solid fat product from a crude semi-solid oil and interesterified palm oil blend (1:1 mass ratio).

Approximately 500 g of semi-solid crude oil containing DHA, produced by Schizochytrium microorganism, was combined with 500 g of interesterified palm oil blend (Cisao 81-36; interesterified product derived from palm oil and palm kernel oil) obtained from AarhusKarlshamn USA Inc. (Port Newark, N.J.). The oil mixture was then heated to 70° C. under nitrogen. About 0.1% (w/w) of 50 wt % citric acid was added and the oil was held at 70° C. under nitrogen for 10 minutes. After 10 minutes, 0.6% (w/w of oil) of caustic solution (50% caustic solution and soft water, 1:3 w/w ratio) was added to the oil and held at 70° C. for 5 minutes. After the 5 minute hold at 70° C., the oil was centrifuged to remove soaps from the oil. Next, Trisyl 600 (0.1% w/w) was added and the temperature was held between 50-55° C. under nitrogen and/or vacuum for 10 minutes. Subsequently, Tonsil Supreme FF bleaching clay (0.5% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes with 3% sparge steam. After deodorization, antioxidants were added. This yields a homogenous product that is solid at room temperature. After cooling to 30-40° C., the resulting crystallized fat was transferred to containers and stored. Quality characteristics and physical properties of the final product are given in Table 16.

TABLE 16
Physical and Chemical Properties of the Final Product
from Example 12

| Parameter | Results |
|---|---|
| DHA (mg/g) | 173.5 |
| PV (meq/kg) | 0.6 |
| p-AV | 4.2 |
| FFA (%) | 0.05 |
| Acid Value (mg KOH/g) | 0.08 |
| Saponification Value | 188.9 |
| Iodine Value | 139.8 |
| Wiley Melting Point (° C.) | 45.7 |
| Solid Fat Content (%): | |
| 10° C. | 33.0 |
| 21.1° C. | 17.7 |
| 26.7° C. | 12.5 |
| 33.3° C. | 8.3 |
| 37.8° C. | 6.6 |
| Fatty Acid Profile (% of total fatty acids): | |
| 12:0 | 0.3 |
| 14:0 | 6.0 |
| 15:1 | 0.4 |
| 16:0 | 38.2 |
| 16:1 | 0.2 |
| 18:0 | 2.7 |
| 18:1n-9 | 20.3 |
| 18:1n-7 | 0.4 |
| 18:2n-6 | 4.9 |
| 20:0 | 0.3 |
| 20:4n-6 | 1.0 |
| 20:5n-3 | 0.7 |
| 22:5n-6 | 6.8 |
| 22:6n-3 | 17.5 |

### Example 13

The following Example shows a process for forming a solid fat product via interesterification of semi-solid crude oil with DHA-stearin (1:1 mass ratio).

Approximately 300 g of crude DHA-stearin, produced by Schizochytrium microorganism containing DHA as a by-product from the winterization process, was vacuum-filtered to remove the filter aid introduced by the winterization process. Approximately 300 g of semi-solid crude oil produced by Schizochytrium microorganism containing DHA was mixed with 0.2% (w/w of oil) Celpure filter aid and vacuum-filtered to remove the moisture from the oil. Approximately 225 g of filtered DHA-stearin was then combined with 225 g of filtered semi-solid crude oil produced by Schizochytrium microorganism containing DHA. The oil mixture was then heated to 9° C. under vacuum and held for 30 minutes under full vacuum. After 30 minutes, the oil mixture was cooled to 80° C. At this time, 1.5% (w/w of oil) of sodium ethoxide solution (21 wt. % solution in denatured ethanol; 6.75 g) was added to the oil and held at 80° C. for 30 minutes under nitrogen. Next, 3% (w/w) water, pre-heated to 80° C., was added and mixed for 5 minutes. The oil mixture was then centrifuged to remove soaps from the oil. Next, Trisyl 600 (0.5% w/w) was added and the temperature was held between 50-55° C. under nitrogen for 15 minutes. Subsequently, Tonsil Supreme FF bleaching clay (1.5% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes. After deodorization, antioxidants were added. This yields a homogenous product that is solid at room temperature. After cooling to 30-40° C., the resulting crystallized fat was transferred to containers and stored. Quality characteristics and physical properties of the final product are given in Table 17.

TABLE 17
Physical and Chemical Properties of the Final Product
from Example 13

| Parameter | Results |
|---|---|
| DHA (mg/g) | 346.1 |
| PV (meq/kg) | 0.0 |
| p-AV | 0.7 |
| FFA (%) | 0.06 |
| Rancimat Value (h) | 17.0 |
| Trans Fatty Acids (%) | Not detected |
| Elemental Analyses (mg/kg): | |
| As | <0.02 |
| Cu | <0.2 |
| Fe | <0.5 |
| Pb | <0.02 |
| Hg | <0.01 |
| Solid Fat Content (%): | |
| 10° C. | 12.1 |
| 21.1° C. | 9.9 |
| 26.7° C. | 7.4 |
| 33.3° C. | 3.9 |
| 37.8° C. | 1.3 |
| Fatty Acid Profile (% of total fatty acids): | |
| 12:0 | 0.3 |
| 14:0 | 11.8 |
| 15:1 | 0.4 |
| 16:0 | 28.5 |
| 16:1 | 0.3 |
| 18:0 | 0.7 |
| 18:1n-9 | 0.5 |
| 18:1n-7 | 0.2 |
| 18:2n-6 | 0.2 |
| 20:0 | 0.2 |
| 20:3n-6 | 0.4 |
| 20:4n-6 | 1.7 |
| 20:5n-3 | 1.0 |
| 22:5n-6 | 14.2 |
| 22:6n-3 | 38.1 |

### Example 14

The following Example shows a process for forming a solid fat product via interesterification of an oil blend.

Approximately 180 g of deodorized semi-solid oil and 24 g of deodorized liquid oil produced by Schizochytrium microorganism containing DHA were combined with 48 g of deodorized palm oil and 48 g of deodorized palm stearin. The oil mixture was then heated to 90-110° C. under vacuum and held for 30-120 minutes under full vacuum. After 30-120 minutes, the oil mixture was cooled to 80-100° C. At this time, 1.0-1.5% (w/w of oil) of sodium ethoxide solution (21 wt. % solution in denatured ethanol) was added to the oil and held at 80-100° C. for 30 minutes under nitrogen. Next, 3% (w/w) water, pre-heated to 80-100° C., was added and mixed for 5-10 minutes. The oil mixture was then centrifuged to remove soaps from the oil.

Next, Trisyl 600 (0.5% w/w) was added and the temperature was held between 50-55° C. under nitrogen for 15 minutes. Subsequently, Tonsil Supreme FF bleaching clay (1.5% w/w) was added and the oil was heated to 9° C. and held under vacuum (>24″ Hg) for 15-30 minutes. Celpure (0.1% w/w) was then added and the oil was filtered under vacuum. The oil was then deodorized at 210° C. for 30 minutes. After deodorization, antioxidants were added. This yields a homogenous product that is solid at room temperature. After cooling to 30-35° C., the resulting crystallized fat was transferred to containers and stored. Quality characteristics and physical properties of the final product are given in Table 18.

TABLE 18
Physical and Chemical Properties of the Final Product
from Example 14

| Parameter | Results |
|---|---|
| DHA (mg/g) | 217.0 |
| PV (meq/kg) | 1.0 |
| p-AV | 2.0 |
| FFA (%) | 0.1 |
| Trans Fatty Acids (%) | Not detected |
| Melting Point (° C.) | 36.0 |
| Solid Fat Content (%): | |
| 10° C. | 20.0 |
| 21.1° C. | 11.7 |
| 26.7° C. | 8.3 |
| 33.3° C. | 4.4 |
| 37.8° C. | 2.4 |
| Fatty Acid Profile (% of total fatty acids): | |
| 12:0 | 0.5 |
| 14:0 | 8.8 |
| 16:0 | 34.6 |
| 16:1 | 0.3 |
| 18:0 | 2.3 |
| 18:1n-9 | 13.9 |
| 18:1n-7 | 0.3 |
| 18:2n-6 | 3.0 |
| 18:3n-6 | 0.1 |
| 18:3n-3 | 0.1 |
| 20:0 | 0.3 |
| 20:3n-6 | 0.2 |
| 20:4n-6 | 1.3 |
| 20:5n-3 | 0.8 |
| 22:5n-6 | 8.8 |
| 22:6n-3 | 23.7 |

### Example 15

The following Example shows a process for forming a solid fat product via physical blending of deodorized semi-solid oil with deodorized palm stearin (4:1 mass ratio).

Approximately 160 g of deodorized semi-solid oil produced by Schizochytrium microorganism containing DHA was combined with 40 g of deodorized palm stearin. The oil mixture was then heated to 65° C. and agitated for 15 minutes. After 15 minutes, the oil mixture was cooled to 30-35° C. This yields a homogenous product that is solid at room temperature. After cooling to 30-35° C., the resulting crystallized fat was transferred to containers and stored. Quality characteristics and physical properties of the final product are given in Table 19.

TABLE 19
Physical and Chemical Properties of the Final Product
from Example 15

| Parameter | Results |
|---|---|
| DHA (mg/g) | 260.0 |
| PV (meq/kg) | 0.3 |
| p-AV | 4.3 |
| FFA (%) | 0.08 |
| Melting Point (° C.) | 45.7 |
| Solid Fat Content (%): | |
| 10° C. | 25.1 |
| 21.1° C. | 19.2 |
| 26.7° C. | 15.4 |
| 33.3° C. | 11.1 |
| 37.8° C. | 8.5 |

### Example 16

The following Example shows a process for forming a solid fat product via physical blending of deodorized semi-solid oil with deodorized palm stearin (5:1 mass ratio).

Approximately 250 g of deodorized semi-solid oil produced by Schizochytrium microorganism containing DHA was combined with 50 g of deodorized palm stearin. The oil mixture was then heated to 65° C. and agitated for 15 minutes. After 15 minutes, the oil mixture was cooled to 30-35° C. This yields a homogenous product that is solid at room temperature. After cooling to 30-35° C., the resulting crystallized fat was transferred to containers and stored. Quality characteristics and physical properties of the final product are given in Table 20.

TABLE 20
Physical and Chemical Properties of the Final Product
from Example 16

| Parameter | Results |
|---|---|
| DHA (mg/g) | 286.3 |
| PV (meq/kg) | 0.4 |
| p-AV | 2.8 |
| FFA (%) | 0.2 |
| Acid Value (mg KOH/g) | 0.3 |
| Wiley Melting Point (° C.) | 43.0 |
| Iodine Value | 210.8 |
| Saponification Value | 181.9 |
| Dropping Point (° C.) | 38.3 |
| Congeal Point (° C.) | 33.0 |
| Solid Fat Content (%): | |
| 10° C. | 19.1 |
| 21.1° C. | 14.1 |
| 26.7° C. | 11.0 |
| 33.3° C. | 8.0 |
| 37.8° C. | 5.6 |
| Fatty Acid Profile (% of total fatty acids): | |
| 12:0 | 0.4 |
| 14:0 | 9.3 |
| 16:0 | 32.8 |
| 16:1 | 0.4 |
| 18:0 | 1.7 |
| 18:1n-9 | 7.8 |
| 18:1n-7 | 0.3 |
| 18:2n-6 | 1.6 |
| 20:0 | 0.2 |
| 20:3n-6 | 0.3 |
| 20:4n-6 | 1.7 |
| 20:5n-3 | 1.2 |
| 22:5n-6 | 11.4 |
| 22:6n-3 | 29.5 |

### Example 17

The following Example shows a process for forming a solid fat product via physical blending of deodorized semi-solid oil with deodorized palm kernel stearin (5:1 mass ratio).

Approximately 250 g of deodorized semi-solid oil produced by Schizochytrium microorganism containing DHA was combined with 50 g of deodorized palm kernel stearin. The oil mixture was then heated to 60° C. and agitated for 15 minutes. After 15 minutes, the oil mixture was cooled to 30-35° C. This yields a homogenous product that is solid at room temperature. After cooling to 30-35° C., the resulting crystallized fat was transferred to containers and stored. Quality characteristics and physical properties of the final product are given in Table 21.

TABLE 21
Physical and Chemical Properties of the Final Product
from Example 17

| Parameter | Results |
|---|---|
| DHA (mg/g) | 289.9 |
| PV (meq/kg) | 0.0 |
| p-AV | 2.5 |
| FFA (%) | 0.1 |
| Acid Value (mg KOH/g) | 0.3 |
| Wiley Melting Point (° C.) | 35.0 |
| Iodine Value | 205.2 |
| Saponification Value | 190.2 |
| Dropping Point (° C.) | 33.3 |
| Congeal Point (° C.) | 26.5 |
| Solid Fat Content (%): | |
| 10° C. | 21.8 |
| 21.1° C. | 11.5 |
| 26.7° C. | 6.4 |
| 33.3° C. | 3.1 |
| 37.8° C. | 0.5 |
| Fatty Acid Profile (% of total fatty acids): | |
| 12:0 | 9.4 |
| 14:0 | 13.7 |
| 16:0 | 24.6 |
| 16:1 | 0.3 |
| 18:0 | 1.0 |
| 18:1n-9 | 3.3 |
| 18:2n-6 | 0.6 |
| 20:3n-6 | 0.3 |
| 20:4n-6 | 1.6 |
| 20:5n-3 | 0.8 |
| 22:5n-6 | 12.0 |
| 22:6n-3 | 30.3 |

### Example 18

The following Example shows a process for forming a solid fat product via physical blending of deodorized semi-solid oil with deodorized palm kernel stearin (9:1 mass ratio).

Approximately 900 g of deodorized semi-solid oil produced by Schizochytrium microorganism containing DHA was combined with 100 g of deodorized palm kernel stearin. The oil mixture was then heated to 60° C. and agitated for 15 minutes. After 15 minutes, the oil mixture was cooled to 30-35° C. This yields a homogenous product that is solid at room temperature. After cooling to 30-35° C., the resulting crystallized fat was transferred to containers and stored. Quality characteristics and physical properties of the final product are given in Table 22.

TABLE 22
Physical and Chemical Properties of the Final Product
from Example 18

| Parameter | Results |
|---|---|
| DHA (mg/g) | 308.4 |
| PV (meq/kg) | 0.9 |
| p-AV | 3.7 |
| FFA (%) | 0.07 |
| Acid Value (mg KOH/g) | 0.06 |
| Wiley Melting Point (° C.) | 35.8 |
| Iodine Value | 219.6 |
| Saponification Value | 187.6 |
| Solid Fat Content (%): | |
| 10° C. | 14.0 |
| 21.1° C. | 7.7 |
| 26.7° C. | 5.2 |
| 33.3° C. | 2.5 |
| 37.8° C. | 0.8 |
| Fatty Acid Profile (% of total fatty acids): | |
| 12:0 | 5.9 |
| 14:0 | 12.8 |
| 16:0 | 25.6 |
| 16:1 | 0.3 |
| 18:0 | 0.9 |
| 18:1n-9 | 2.7 |
| 18:2n-6 | 0.5 |
| 20:3n-6 | 0.4 |
| 20:4n-6 | 1.9 |
| 20:5n-3 | 1.4 |
| 22:5n-6 | 12.8 |
| 22:6n-3 | 32.9 |

### Example 19

The following Example shows a process for forming a solid fat product via physical blending of deodorized semi-solid oil with deodorized Cisao 81-36 (interesterified palm oil blend) at 9:1 mass ratio.

Approximately 900 g of deodorized semi-solid oil produced by Schizochytrium microorganism containing DHA was combined with 100 g of deodorized Cisao 81-36. The oil mixture was then heated to 60° C. and agitated for 15 minutes. After 15 minutes, the oil mixture was cooled to 30-35° C. This yields a homogenous product that is solid at room temperature. After cooling to 30-35° C., the resulting crystallized fat was transferred to containers and stored. Quality characteristics and physical properties of the final product are given in Table 23.

TABLE 23
Physical and Chemical Properties of the Final Product
from Example 19

| Parameter | Results |
|---|---|
| DHA (mg/g) | 311.2 |
| PV (meq/kg) | 0.6 |
| p-AV | 3.5 |
| FFA (%) | 0.04 |
| Acid Value (mg KOH/g) | 0.06 |
| Wiley Melting Point (° C.) | 34.3 |
| Iodine Value | 219.5 |
| Saponification Value | 187.5 |
| Solid Fat Content (%): | |
| 10° C. | 13.3 |
| 21.1° C. | 9.2 |
| 26.7° C. | 6.8 |
| 33.3° C. | 4.1 |
| 37.8° C. | 2.6 |
| Fatty Acid Profile (% of total fatty acids): | |
| 12:0 | 0.4 |
| 14:0 | 10.4 |
| 16:0 | 29.4 |
| 16:1 | 0.3 |
| 18:0 | 1.2 |
| 18:1n-9 | 5.9 |
| 18:2n-6 | 1.2 |
| 20:3n-6 | 0.4 |
| 20:4n-6 | 1.9 |
| 20:5n-3 | 1.4 |
| 22:5n-6 | 12.8 |
| 22:6n-3 | 32.8 |

### Example 20

The following Example shows a process for forming a solid fat product via physical blending of different oils.

Approximately 120 g of deodorized semi-solid oil and 16 g of deodorized liquid oil produced by Schizochytrium microorganism containing DHA were combined with 32 g of deodorized palm oil and 32 g of deodorized palm stearin. The oil mixture was then heated to 70° C. and agitated for 15 minutes. After 15 minutes, the oil mixture was cooled to 30-35° C. This yields a homogenous product that is solid at room temperature. After cooling to 30-35° C., the resulting crystallized fat was transferred to containers and stored. Quality characteristics and physical properties of the final product are given in Table 24.

TABLE 24
Physical and Chemical Properties of the Final Product
from Example 21

| Parameter | Results |
|---|---|
| DHA (mg/g) | 223.5 |
| PV (meq/kg) | 2.2 |
| p-AV | 0.0 |
| FFA (%) | 0.07 |
| Solid Fat Content (%): | |
| 10° C. | 26.9 |
| 21.1° C. | 16.7 |
| 26.7° C. | 13.0 |
| 33.3° C. | 9.1 |
| 37.8° C. | 6.6 |
| Fatty Acid Profile (% of total fatty acids): | |
| 12:0 | 0.5 |
| 14:0 | 8.9 |
| 15:1 | 0.3 |
| 16:0 | 34.8 |
| 16:1 | 0.3 |
| 18:0 | 2.3 |
| 18:1n-9 | 13.8 |
| 18:2n-6 | 3.0 |
| 20:0 | 0.3 |
| 20:4n-6 | 1.3 |
| 20:5n-3 | 0.0 |
| 22:5n-6 | 8.8 |
| 22:6n-3 | 23.5 |

### Example 21

The following Example shows a bench-scale process for forming a solid fat product from crude fish oil and palm oil (1:3 mass ratio).

Approximately 75 g of crude menhaden oil and 225 g of crude palm oil were combined. The oil mixture was then heated to 50-55° C. under nitrogen. About 0.2% (w/w of oil) of 50 wt % citric acid was added to the oil and the oil was held at 50-55° C. under nitrogen for 15 minutes. After 15 minutes, the oil mixture was heated to 65-70° C. At this time, 5.0% (w/w of oil) of caustic solution (50% caustic solution and soft water, 1:3 w/w ratio) was added to the oil and held at 65-70° C. for 15 minutes. After the 15 minute hold at 65-70° C., oil mixture was centrifuged to remove soaps from the oil. Next, Trisyl 600 (0.1% w/w) was added and the temperature was held between 50-55° C. under nitrogen for 15 minutes. Subsequently, Tonsil Supreme FF bleaching clay (1.0% w/w) was added and the oil was heated to 90° C. and held under vacuum (>24″ Hg) for 15 minutes. Celpure (0.1% w/w) was then added and the oil was filtered under vacuum. The oil was then deodorized at 210° C. for 30 minutes. After deodorization, antioxidants were added. This yields a homogenous product that is solid at room temperature. After cooling to 30-35° C., the resulting crystallized fat was transferred to containers and stored. Quality characteristics and physical properties of the final product are given in Table 25.

TABLE 25
Physical and Chemical Properties of the Final Product
from Example 21

| Parameter | Results |
|---|---|
| DHA (mg/g) | 18.3 |
| PV (meq/kg) | 0.26 |
| p-AV | 3.3 |
| FFA (%) | 0.3 |
| Wiley Melting Point (° C.) | 32.8 |
| Iodine Value | 84.5 |
| Saponification Value | 197.7 |
| Solid Fat Content (%): | |
| 10° C. | 33.6 |
| 21.1° C. | 9.9 |
| 26.7° C. | 5.1 |
| 33.3° C. | 2.0 |
| 37.8° C. | 0.9 |
| Fatty Acid Profile (% of total fatty acids): | |
| 12:0 | 0.5 |
| 14:0 | 3.1 |
| 16:0 | 35.0 |
| 16:1 | 2.9 |
| 18:0 | 4.4 |
| 18:1n-9 | 33.9 |
| 18:1n-7 | 1.2 |
| 18:2n-6 | 7.5 |
| 18:3n-6 | 0.1 |
| 18:3n-3 | 0.4 |
| 20:0 | 0.4 |
| 20:3n-6 | 0.0 |
| 20:4n-6 | 0.3 |
| 20:5n-3 | 3.7 |
| 22:5n-3 | 0.6 |
| 22:6n-3 | 1.8 |

The principles, preferred embodiments and modes of operation of the present invention have been described in the foregoing specification. When sources and amounts or ranges of the fatty acids and other ingredients are used herein, all combinations and subcombinations and specific embodiments therein are intended to be included. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the foregoing best mode of carrying out the invention should be considered exemplary in nature and not as limiting to the scope and spirit of the invention as set forth in the appended claims.

What is claimed is:

1. A method for producing a solid fat composition comprising:
a) mixing an oil comprising saturated fat with an oil comprising at least one LC-PLJFA to form a mixture; and
b) solidifying the mixture to form a solid fat composition, wherein no exogenous emulsifier is added in producing the solid fat composition.

2. The method of claim 1, wherein the oil comprising saturated fat is selected from the group consisting of microbial stearin, unfractionated palm oil, palm olein, palm stearin, palm mid fraction, unfractionated palm kernel oil, palm kernel olein, palm kernel stearin, unfractionated cotton seed oil, cotton seed olein, cotton seed stearin, coconut oil, unfractionated shea butter oil, shea butter stearin, interesterified palm oil blend, interesterified cotton seed oil blend, fish oil stearin, and combinations thereof.

3. The method of claim 1, wherein the oil comprising at least one LC-PUFA is unwinterized.

4. The method of claim 1, wherein the oil comprising at least one LC-PUFA comprises saturated fat.

5. The method of claim 1, wherein the oil comprising at least one LC-PUFA comprises between about 5% to about 70% by weight of at least one LC-PUFA selected from the group consisting of docosahexaenoic acid, omega-3 or omega-6 docosapentaenoic acid, arachidonic acid, and eicosapentaenoic acid.

6. The method of claim 1, wherein the oil comprising saturated fat and the oil comprising at least one LC-PUFA are not heated prior to the mixing step.

7. The method of claim 1, wherein the solid fat composition is selected from the group consisting of a food product, a nutritional product and a pharmaceutical product.

8. The method of claim 1, wherein the ratio of the oil comprising at least one LC-PUFA to the oil comprising saturated fat is from about 1:9 to about 9:1 by weight.

9. The method of claim 1, further comprising deodorizing the mixture.

10. The method of claim 1, further comprising interesterifying the mixture.

11. The method of claim 1, wherein the oil comprising at least one LC-PUFA is from a source selected from the group consisting of a microbial source, a plant source and an animal source.

12. The method of claim 1, wherein the oil comprising at least one LC-PUFA is from a microbial source.

13. A solid fat composition comprising a mixture of an oil comprising saturated fat and an oil comprising at least one LC-PUFA, wherein the mixture is solid at room temperature, and wherein the mixture contains no exogenous emulsifier.

14. The solid fat composition of claim 13, wherein the oil comprising saturated fat is selected from the group consisting of microbial stearin, unfractionated palm oil, palm olein, palm stearin, palm mid fraction, unfractionated palm kernel oil, palm kernel olein, palm kernel stearin, unfractionated cotton seed oil, cotton seed olein, cotton seed stearin, coconut oil, unfractionated shea butter oil, shea butter stearin, interesterified palm oil blend, interesterified cotton seed oil blend, fish oil stearin, and combinations thereof.

15. The solid fat composition of claim 13, wherein the oil comprising at least one LC-PUFA is unwinterized.

16. The solid fat composition of claim 13, wherein the oil comprising at least one LC-PUFA comprises saturated fat.

17. The solid fat composition of claim 13, wherein the oil comprising at least one LC-PUFA comprises between about 5% to about 70% by weight of at least one LC-PUFA selected from the group consisting of docosahexaenoic acid, omega-3 or omega-6 docosapentaenoic acid, arachidonic acid, and eicosapentaenoic acid

18. The solid fat composition of claim 13, wherein the solid fat composition is free of trans-fatty acids.

19. The solid fat composition of claim 13, wherein the ratio of the oil comprising at least one LC-PUFA to the oil comprising saturated fat is from about 1:9 to about 9:1 by weight.

20. The solid fat composition of claim 13, wherein the solid fat composition is selected from the group consisting of a food product, a nutritional product and a pharmaceutical product.

21. The solid fat composition of claim 13, wherein the oil comprising at least one LC-PUFA is from a source selected from the group consisting of a microbial source, a plant source and an animal source.

22. The solid fat composition of claim 13, wherein the oil comprising at least one LC-PUFA is from a microbial source.

23. A method for producing a solid fat composition comprising:
a) mixing a stearin comprising at least one LC-PUFA with a second oil comprising saturated fat to form a mixture; and
b) solidifying the mixture to form a solid fat composition.

24. The method of claim 23, wherein no exogenous emulsifier is added in producing said solid fat composition.

25. The method of claim 23, wherein the stearin is selected from the group consisting of microbial stearin, fish oil stearin, palm stearin, palm kernel stearin, cotton seed stearin, shea butter stearin, and combinations thereof.

26. The method of claim 23, wherein the second oil comprising saturated fat is selected from the group consisting of unfractionated palm oil, palm olein, unfractionated palm kernel oil, palm kernel olein, palm mid fraction, coconut oil, unfractionated shea butter oil, unfractionated cotton seed oil, cotton seed olein, interesterified palm oil blend, interesterified cotton seed oil blend, and combinations thereof.

27. A solid fat composition comprising a mixture of a stearin composition comprising at least one LC-PUFA and a second oil comprising saturated fat, wherein the composition is solid at room temperature.

28. The solid fat composition of claim 27, wherein the stearin is selected from the group consisting of microbial stearin, fish oil stearin, palm stearin, palm kernel stearin, cotton seed stearin, shea butter stearin, and combinations thereof.

29. The solid fat composition of claim 27, wherein the second oil comprising saturated fat is selected from the group consisting of unfractionated palm oil, palm olein, unfractionated palm kernel oil, palm kernel olein, palm mid fraction, coconut oil, unfractionated shea butter oil, shea butter stearin, unfractionated cotton seed oil, cotton seed olein, interesterified palm oil blend, interesterified cotton seed oil blend, and combinations thereof.
